(19) United States
(12) Patent Application Publication
(10) Pub. No.: US 2018/0320233 A1
(43) Pub. Date: Nov. 8, 2018

(54) GENOMICS-BASED, TECHNOLOGY-DRIVEN MEDICINE PLATFORMS, SYSTEMS, MEDIA, AND METHODS

(71) Applicant: Human Longevity, Inc., San Diego, CA (US)

(72) Inventor: Bradley A. PERKINS, San Diego, CA (US)

(21) Appl. No.: 15/968,571

(22) Filed: May 1, 2018

Related U.S. Application Data
(60) Provisional application No. 62/500,426, filed on May 2, 2017.

(51) Int. Cl. C12Q 1/6883; G06F 19/22; G16H 10/40; G16H 50/30; G16H 50/20

(52) U.S. Cl. CPC C12Q 1/6883; G06F 19/22; G16H 50/20; G16H 50/30; G16H 10/40

(57) ABSTRACT

Disclosed are methods, media, and systems for detecting an undiagnosed medical condition by acquiring a plurality of health metrics of the individual, wherein at least one of the plurality of health metrics comprises nucleotide sequence data; implementing a genetic risk rule that defines a genetic risk for the undiagnosed medical condition; implementing a non-genetic risk rule that defines a non-genetic risk for the undiagnosed medical condition; and generating a confidence score for the undiagnosed medical condition that comprises a function of the genetic risk rule and the non-genetic risk rule.

### APPLICATIONS FOR CLAIM OF PRIORITY

This application claims the benefit of priority under 35 U.S.C. § 119 from U.S. Provisional Patent Application 62/500,426 filed May 2, 2017. The disclosure of the above identified application is incorporated herein by reference in its entirety as if set forth in full.

### BACKGROUND

Progress in science and technology with shifting epidemiology and demographics are creating the capabilities and demand for alternatives to symptom-driven medical models. Reducing age-related chronic diseases associated with premature mortality among adults is an urgent priority requiring new approaches and technologies.

The near-doubling of average human life expectancy over the last 150 years is a tribute to scientific advancements in medicine and public health. Most of this success—though not all—is the result of progress in control and prevention of infectious diseases particularly among young children. Eighty-five percent of children born now in the US are expected to live to 65 years of age; at least 42% will likely celebrate an 85th birthday. Because of these successes most of humanity is facing a daunting and costly new medical challenge in the form of age-related chronic diseases.

Most age-related chronic diseases have heritability, are often slowly progressive with symptom-free onset, and are associated with common risk factors. In 2015, the estimated US cumulative mortality risk among males 50 to 74 years of age was 39%; for women, the risk was lower but still substantial at 24%. The causes of these deaths are similar across genders with neoplasms and cardiovascular disease accounting for about one-third each, and diabetes and related conditions, respiratory, cirrhosis and other liver diseases, and neurologic disorders accounting for the remaining one-third.

### SUMMARY

The vast majority of primary medical interventions are informed by and implemented upon population based studies. This is a problem since a single individual cannot be adequately represented by an entire population due to genetic and environmental heterogeneity within the population.

Few examples demonstrate how genomics might be proactively incorporated into new models for medical practice, and what infrastructure will be needed to support data generation and use. The methods described herein allow the integration of disparate orthogonal health data in a quantitative way to enable disease diagnosis and the determination of an optimal treatment plan. These methods are also useful for determining an increased risk of a future diagnosis of a disease, and importantly they allow for the identification of sub symptomatic diseases allowing earlier treatment and intervention in order to increase positive health outcomes.

Genomics as currently applied has been disappointing in its ability to unravel the “missing heritability” of most age-related chronic diseases, and other common diseases. This is rapidly changing as a result of public and private efforts to expand sequencing. First, we are increasingly finding and seeing supporting evidence for the increasing recognition of rare variants with large effect sizes. Combining this with advancements in monogenic and polygenic methodologies to assess causation including Mendelian randomization methods, extension of genome-wide association study to create hazard models, and continued exploration of pleiotropy, increase clinical utility. Second, increasingly detailed mapping of molecular pathways and mechanisms associated with diseases and risk factors provide a much needed improved capability to link genotype and phenotype data. Described herein, we demonstrate the use of global metabolomics in mapping to genomic defects. This can significantly strengthen with additional experience and automation. Thirdly, we quantitatively integrate genomics with other clinical data, particularly advanced imaging data, to create point-of-care clinical decision support. For example, if one queries more than 40,000 genomes (individuals and families), genotype-phenotype associations can be described with millisecond response times. The methods described herein prioritize individual opportunities for tertiary (disease treatment), secondary (risk factor control), and primary prevention using human- and machine-driven feature extraction.

The value of evolving medical practice from disease diagnosis to risk detection is supported by the study in Example 1. For example, we recommended follow-up imaging studies for slightly more than one-third of our study participants. Some of this is the nature of screening, which drives need for more definitive imaging studies better suited to specific abnormalities. Other instances of referral were intended to identify change over a specified time period which might be suggestive of cancer such as finding a cystic pancreatic lesion or instability of a vascular lesion such an intracranial aneurysm. In some instances, we don't know enough to confidently predict the natural course of these findings, and as a result may cause unnecessary anxiety and unneeded surgery. However, the life-threatening consequences and relatively high prevalence of diseases associated with these lesions suggests that early recognition is likely to be beneficial for most individuals. Expansion of some or all of our approach to broader populations requires the methods described herein.

The methods, computer media, and systems described herein deploy genome sequencing (e.g., whole genome sequencing, exosome sequencing, SNP typing) in combination with one or more other routine and advanced diagnostic technologies including: microbiome sequencing (e.g., gut or dermal microbiome); global metabolomics; 3D/4D imaging focusing on non-contrast whole-body magnetic resonance imaging and echocardiogram; 2-week cardiac monitoring; and functional neurologic testing to detect risk for age-related chronic diseases.

In a certain aspect, described herein, is a method of detecting an undiagnosed medical condition comprising: acquiring a plurality of health metrics of the individual, wherein at least one of the plurality of health metrics comprises nucleotide sequence data; implementing a genetic risk rule that defines a genetic risk for the undiagnosed medical condition; implementing a non-genetic risk rule that defines a non-genetic risk for the undiagnosed medical condition; and generating a confidence score for the undiagnosed medical condition that comprises a function of the genetic risk rule and the non-genetic risk rule. In certain embodiments, the undiagnosed medical condition is an increased likelihood of developing a medical condition. In certain embodiments, the medical condition comprises Parkinson's disease, Alzheimer's disease, ischemic heart disease, hyperlipidemia, high blood pressure, cardiac arrhythmia, long QT syndrome, insulin resistance, Type II diabetes, non-alcoholic fatty liver disease, cirrhosis of the liver, kidney failure, heart failure, depression, bipolar disorder, schizophrenia, or a cancer. In certain embodiments, the cancer comprises breast cancer, prostate cancer, lung cancer, melanoma, pancreatic cancer, kidney cancer, skin cancer, bladder cancer, ovarian cancer, cervical cancer, colon cancer, a leukemia, a lymphoma, head and neck cancer, or brain cancer. In certain embodiments, the nucleotide sequence data comprises DNA sequence data. In certain embodiments, the nucleotide sequence data comprises a list of nucleotide sequence variants compared to a reference genome. In certain embodiments, the plurality of health metrics further comprises a phenotypic measurement, a family medical history, a personal medical history, or a gut microbiome assessment. In certain embodiments, the phenotypic measurement comprises a clinical measurement or a clinical laboratory test. In certain embodiments, the clinical measurement or the clinical laboratory test comprises a sleep apnea score, cognitive assessment, neurological test, quantitative Neuro imaging, balance assessment, gait assessment, weight, height, systolic blood pressure, diastolic blood pressure, resting pulse rate, cardiac rhythm monitoring, electrocardiogram, blood lipid levels, blood glucose level, oral glucose tolerance test, blood insulin level, body fat measurement, or whole body MRI. In certain embodiments, the whole body MRI comprises an estimate of total body fat mass or percentage, subcutaneous fat mass or percentage, visceral fat mass or percentage, muscle mass or percentage, liver fat mass or percentage, brain volume, or hippocampal volume. In certain embodiments, the genetic risk rule comprises ranking a nucleotide sequence variant based upon a score reflecting a pathogenicity of the nucleotide sequence for the undiagnosed medical condition. In certain embodiments, the pathogenicity of the nucleotide sequence for the undiagnosed medical condition is previously determined using a genome wide association study or hazard score associated therewith, presence in ClinVar database, or presence in a gene known or suspected to be causative for the undiagnosed medical condition. In certain embodiments, the second set of rules comprises ranking the non-genetic risk for the undiagnosed medical condition comprises ranking the phenotypic measurement against a plurality of phenotypic measurements derived from a population of individuals. In certain embodiments, ranking the non-genetic risk for the undiagnosed medical condition comprises assigning a quantile score to the non-genetic risk for the undiagnosed medical condition. In certain embodiments, ranking the non-genetic risk for the undiagnosed medical condition comprises assigning a quintile score to the non-genetic risk for the undiagnosed medical condition. In certain embodiments, the second set of rules comprises determining an amount of standard deviations the phenotypic measurement is away from a mean level for the undiagnosed medical condition derived from a plurality of phenotypic measurements derived from a population of individuals. In certain embodiments, the amount of standard deviations is greater than 2. In certain embodiments, the confidence score for the undiagnosed medical condition that comprises a function of the genetic risk rule and the non-genetic risk rule is more accurate than a confidence score for either the genetic risk rule or the non-genetic risk rule alone. In certain embodiments, the method further comprises delivering a report of the confidence score for the undiagnosed medical condition to a health care provider. In certain embodiments, the method further comprises delivering a report of the confidence score for the undiagnosed medical condition to an individual. In certain embodiments, the undiagnosed medical condition comprises a plurality of undiagnosed medical conditions. In certain embodiments, a non-transitory computer-readable storage media is encoded with a computer program including instructions executable by a processor to create a program to detect an undiagnosed medical condition according to the methods described herein.

### BRIEF DESCRIPTION OF THE DRAWINGS

A better understanding of the features and advantages of the embodiments in the present disclosure will be obtained by reference to the following detailed description that sets forth illustrative embodiments and the accompanying drawings of which:

FIG. 1 illustrates a non-limiting algorithm for defining a genetic risk;

FIG. 2 shows a non-limiting example of a digital processing device; in this case, a device with one or more CPUs, a memory, a communication interface, and a display;

FIG. 3 shows a non-limiting example of a web/mobile application provision system; in this case, a system providing browser-based and/or native mobile user interfaces;

FIG. 4 shows a non-limiting example of a cloud-based web/mobile application provision system; in this case, a system comprising an elastically load balanced, auto-scaling web server and application server resources as well synchronously replicated databases;

FIG. 5 shows a flow chart of the methodology of the study employed in the Example 1;

FIG. 6 shows a depiction of phenotype/genotype measurements that are integrated in the methods described herein based on study results; and

FIG. 7 shows a flowchart for phenotype/genotype interaction based on study results.

### DETAILED DESCRIPTION

### Certain Definitions

Unless otherwise defined, all technical terms used herein have the same meaning as commonly understood by one of ordinary skill in the art to which the embodiments disclosed herein belongs. As used in this specification and the appended claims, the singular forms “a,” “an,” and “the” include plural references unless the context clearly dictates otherwise. Any reference to “or” herein is intended to encompass “and/or” unless otherwise stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

As used herein “genomic sequence variant” refers to any nucleotide difference in an individual's genome sequence compared to a reference genome or reference sequence. The variant can be a single nucleotide variant (SNV), insertion or deletion (Indel), or translocation. In certain embodiments, the indel comprises more than a single nucleotide. In certain embodiments, a genomic sequence variant excludes mitochondrial deoxyribonucleic acid (DNA) sequences. In certain embodiments, a genomic sequence variant excludes variants found on either of the non-autosomal human X or Y chromosomes. In certain embodiments, the genomic sequence variant is a human genomic sequence variant.

As used herein “reference genome” refers to any standard publicly available reference genome, for example GRCh38, the Genome Reference Consortium human genome (build 38). Alternatively, the reference genome can be one that is constructed de novo from sequencing a plurality of genomes. In certain embodiments, the plurality of genomes is greater than 10,000 different genomes. In certain embodiments, the plurality of genomes is greater than 100,000 different genomes.

As used herein “disease” refers to any cause of mortality or decreased quality of life that is independent of natural cause. Disease include: age-related chronic diseases; accidents and injuries; cancers; autoimmune/inflammatory diseases, infectious diseases, genetic diseases, psychological disease and maternal/fetal health.

### Medical Conditions

The methods, computer media, and systems described herein are useful for diagnosing hidden, latent, or subsymptomatic conditions. Importantly this type of diagnosis is beyond the current reach of physicians relying solely on in-office examination and laboratory testing. In addition to a diagnosis of active disease, the methods described herein are useful for determining an increased risk of developing a disease. The diseases diagnosed or identified as high risk include, but are not limited to: age-related chronic diseases, such as, Parkinson's disease, Alzheimer's disease, dementia, ischemic heart disease, hyperlipidemia, high blood pressure, cardiac arrhythmia, long QT syndrome, insulin resistance, Type II diabetes, non-alcoholic fatty liver disease, cirrhosis of the liver, liver failure, kidney failure, heart failure, cardiovascular disease, congestive heart failure, emphysema, chronic obstructive pulmonary disease; accidents and injuries, such as, increased risk of alcohol related injuries, increased risk of injury due to voluntary intoxication with drugs or alcohol, self-inflicted injuries, suicide attempts, occupational injuries, sports/fitness-related injuries; infectious diseases, such as, bacterial, viral or parasitic infections, fungal infections, genetic diseases, such as inborn errors of metabolism, X-linked recessive disorders, autosomal dominant disorders, immunodeficiency, cystic fibrosis, enzyme deficiency; psychological disease, such as, depression, bipolar disorder, schizophrenia, mania, anxiety disorder; maternal/fetal health, such as, gestational diabetes, preeclampsia, miscarriage, or sudden infant death syndrome.

The methods, computer media, and systems described herein are useful for diagnosing hidden, latent, or subsymptomatic cancers. The cancer can comprise a cancer such as: lymphoma/leukemia; head and neck cancer; brain cancer; stomach cancer; pancreatic cancer; colon cancer; liver cancer; renal cancer; breast cancer; prostate cancer; cervical cancer; ovarian cancer; acute lymphoblastic leukemia, adult; acute lymphoblastic leukemia, childhood; acute myeloid leukemia, adult; acute myeloid leukemia, childhood; adreno-cortical carcinoma; AIDS-related cancers; AIDS-related lymphoma; anal cancer; appendix cancer; astrocytomas; atypical teratoid/rhabdoid tumor; basal cell carcinoma; bile duct cancer, extrahepatic; bladder cancer; bone cancer, osteosarcoma and malignant fibrous histiocytoma; brain stem glioma; brain tumor; central Nervous System embryonal tumors; astrocytomas; craniopharyngioma; ependymoblastoma; brain tumor, ependymoma; medulloblastoma; medulloepithelioma; Pineal Parenchymal tumors of Intermediate differentiation; supratentorial primitive neuro ectodermal tumors and pineoblastoma; brain and Spinal cord tumors; breast cancer; breast cancer, male; bronchial tumors; Burkitt lymphoma; carcinoid tumor; central nervous system atypical teratoid/rhabdoid tumor; central nervous system embryonal tumors; central nervous system (CNS) lymphoma, cervical cancer; primary; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; embryonal tumors, central nervous system; endometrial cancer; ependymoblastoma; ependymoma; esophageal cancer; esthesioneuroblastoma; Ewing sarcoma family of tumors; extracranial germ cell tumor; extragonadal germ cell tumor; extrahepatic bile duct cancer; eye cancer, Intraocular melanoma; eye cancer, Retinoblastoma; gallbladder cancer; gastric (stomach) cancer; gastrointestinal carcinoid tumor; gastrointestinal Stromal tumor (GIST); germ cell tumor, extracranial; germ cell tumor, extragonadal; germ cell tumor, ovarian; gestational trophoblastic tumor; glioma; hairy cell leukemia; head and Neck cancer; heart cancer; hepatocellular (liver) cancer, adult (Primary); hepatocellular (liver) cancer; histiocytosis, langerhans cell; Hodgkin lymphoma, adult; Hodgkin lymphoma, childhood; hypopharyngeal cancer; Intraocular melanoma; islet cell tumors (endocrine pancreas); Kaposi Sarcoma; kidney (renal cell) cancer; kidney cancer; langerhans cell histiocytosis; laryngeal cancer; laryngeal cancer, childhood; leukemia, acute lymphoblastic, adult; leukemia, acute lymphoblastic, childhood; leukemia, acute myeloid, adult; leukemia, acute myeloid, childhood; leukemia, chronic lymphocytic; leukemia, chronic myelogenous; leukemia, hairy cell; lip and oral cavity cancer; liver cancer, adult (Primary); liver cancer; lung cancer, non-small cell; lung cancer, small cell; lymphoma, AIDS-related; lymphoma, Burkitt; lymphoma, cutaneous T-cell; lymphoma, Hodgkin, adult; lymphoma, Hodgkin, childhood; lymphoma, non-Hodgkin, adult; lymphoma, non-Hodgkin, childhood; lymphoma, primary central nervous system (CNS); macroglobulinemia, Waldenstrom; malignant fibrous histiocytoma of bone and osteosarcoma; medulloblastoma; medulloepithelioma; melanoma; melanoma, Intraocular (eye); Merkel cell carcinoma; mesothelioma, adult malignant; mesothelioma; metastatic Squamous Neck cancer with occult primary; mouth cancer; multiple endocrine neoplasia Syndrome; multiple myeloma/Plasma cell neoplasm; mycosis fungoides; myelodysplastic Syndromes; myelodysplastic/myeloproliferative neoplasms; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-Hodgkin lymphoma, adult; non-Hodgkin lymphoma, childhood; Non-Small cell lung cancer; oral cancer; oral cavity cancer, lip and; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, Islet cell tumors; papillomatosis; paranasal sinus and nasal cavity cancer; parathyroid cancer; penile cancer; pharyngeal cancer; pineal parenchymal tumors of intermediate differentiation; pituitary tumor; plasma cell neoplasm/multiple myeloma; pleuropulmonary blastoma; Pregnancy and breast cancer; primary central nervous system (CNS) lymphoma; prostate cancer; rectal cancer; renal cell (Kidney) cancer; renal pelvis and ureter, transitional cell cancer; respiratory tract cancer with chromosome 15 changes; retinoblastoma; rhabdomyosarcoma; salivary gland cancer; salivary gland cancer; sarcoma, Ewing sarcoma family of tumors; sarcoma, Kaposi; sarcoma, soft tissue, adult; Sarcoma, soft tissue, childhood; Sarcoma, uterine; Sézary syndrome; skin cancer (non-melanoma); skin cancer; skin cancer (melanoma); skin carcinoma, Merkel cell; small cell lung cancer; small intestine cancer; soft tissue sarcoma, adult; soft tissue sarcoma, childhood; squamous cell carcinoma; squamous neck cancer with occult primary, metastatic; stomach (gastric) cancer; supratentorial primitive neuroectodermal tumors; T-cell lymphoma, cutaneous; testicular cancer; throat cancer; thyoma and thymic carcinoma; thyroid cancer; transitional cell cancer of the renal pelvis and ureter; trophoblastic tumor, gestational; unknown primary site, carcinoma of; ureter and renal pelvis, transitional cell cancer; urethral cancer; uterine cancer, endometrial; uterine sarcoma; uveal melanoma; vaginal cancer; vulvar cancer; Waldenström macroglobulinemia; or Wilm's tumor.

The methods, computer media, and systems described herein are useful for diagnosing hidden, latent, or subsymptomatic autoimmune or inflammatory disorders. The autoimmune or inflammatory can comprise an autoimmune or inflammatory disorder such as: acute optic neuritis, alopecia areata, ankylosing spondylitis, antiphospholipid syndrome, autoimmune Addison's disease, autoimmune diseases of the adrenal gland, arthritis, autoimmune hemolytic anemia, autoimmune hepatitis, autoimmune oophoritis and orchitis, autoimmune thrombocytopenia, Behcet's disease, bullous pemphigoid, bronchiolitis obliterans, cardiomyopathy, celiac sprue- dermatitis, chronic fatigue immune dysfunction syndrome (CFIDS), chronic inflammatory demyelinating polyneuropathy, Crohn's disease, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, inflammatory bowel disease (IBD), juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, anti-glomerular basement membrane disease, antiphospholipid syndrome, autoimmune diseases of the nervous system, familial Mediterranean fever, Lambert-Eaton Myasthenic syndrome, sympathetic ophthalmia, or polyendocrinopathies, or sepsis.

### Nucleotide Sequences

In addition to the phenotypic or clinical measurements gathered from an individual the health metric comprises nucleic acid sequence data from an individual. The nucleic acid sequence can comprise one or more DNA sequences. In certain embodiments, the DNA sequence comprises a sequence for an individual's whole genome. In certain embodiments, the DNA sequence comprises a sequence for only the high confidence regions of an individual's whole genome. For example the high confidence region of an individual's whole genome can be defined by the NA12878 Genome-In-A-Bottle call set (GiaB v2.19). In certain embodiments, the DNA sequence comprises a sequence for greater than 99%, 95%, 90%, 85%, 80%, 75%, 70%, 65%, or 60% of the high confidence region of an individual's whole genome as defined by the GiaB v2.19. The DNA sequence can comprise a sequence of a plurality of contiguous nucleotides from an individual's genome. In certain embodiments, the DNA sequence comprises a sequence of at least 100; 1,000; 10,000; 100,000; or 1,000,000 contiguous nucleotides, including increments therein, from an individual's genome. In certain embodiments, the DNA sequence does not comprise the sequence of ribonucleic acid (RNA). In certain embodiments, the DNA sequence does not comprise the sequence of cDNA generated from ribonucleic acid (RNA). The DNA sequence can be generated from an individual's healthy tissue, semen, blood, plasma, serum, saliva, or stool. Additionally envisioned are DNA sequences generated from a tumor sample whether benign or malignant.

In certain embodiments, DNA sequence data for use with the methods, systems and media, described herein, is generated by any suitable method. The DNA sequence data can be generated by Sanger sequencing or by any next-generation sequencing technology. In certain embodiments, the DNA sequence data is generated, by way of non-limiting example, by pyrosequencing, sequencing by synthesis, sequencing by ligation, ion semiconductor sequencing, or single molecule real time sequencing. In certain embodiments, the DNA sequence data is generated by any technology capable of generating 1 gigabase of nucleotide reads per 24 hour period. In certain embodiments, the DNA sequence data is obtained from a third party or from a contracted provider.

In certain embodiments, the health metric for use with the methods, systems and media, comprises described herein is a plurality of genomic sequence variants (GSV). The genomic sequence variants can be determined de novo during implementation of any of the methods either by comparing to a reference genome or a reference genome constructed on the fly from a plurality of greater than 1,000; 10,000; or 100,000 different genomes, including increments therein. In certain embodiments, GSVs are determined by a third party and received by the party performing the method. In certain embodiments, determining a GSV encompasses receiving a list or file that comprises an individual's GSVs. The health metrics utilize a plurality of GSVs. In some cases greater than 10; 50; 100; 500; 1,000; 10,000; 100,000; or 1,000,000 GSVs, including increments therein when compared to a reference sequence, are utilized for the health metric.

### Genetic Risk Rule

The genomic sequence variants of an individual are used to set that individual's genetic risk. This genetic risk is defined by a genetic risk rule. This genetic risk can be disease specific, for example, diabetes, heart failure, specific cancers, or other mortalities. GSVs are first determined and then their potential pathogenicity is determined. Pathogenicity can be determined by using for example a statistical association of the GSV with a given disease or a given gene involved in a given disease. The pathogenicity can be defined based upon a score such as a CADD score, presence in the NCBI ClinVar database, or by other methods of determining a selection pressure on the specific genomic locus. Methods for defining selective pressure through for example a context dependent tolerability score, an n-variant score, regional variation score or a protein tolerability score can be implemented such as those described in U.S. Provisional Application Ser. No. 62/333,653 or U.S. Provisional Application Ser. No. 62/410,783 and are incorporated by reference herein in their entirety. The genetic risk rule can for example rank pathogenic variants by percentile, quartile, quintile, etc. For example, only the top 99^{th}, 98^{th}, 97^{th}, 96^{th}, 95^{th}, 94^{th}, 93^{th}, 92^{nd}, 91^{st}, 90^{th}, 80^{th}, 70^{th}, 60^{th}, or 50^{th} percentile, including increments therein, may be used when determining a genetic risk.

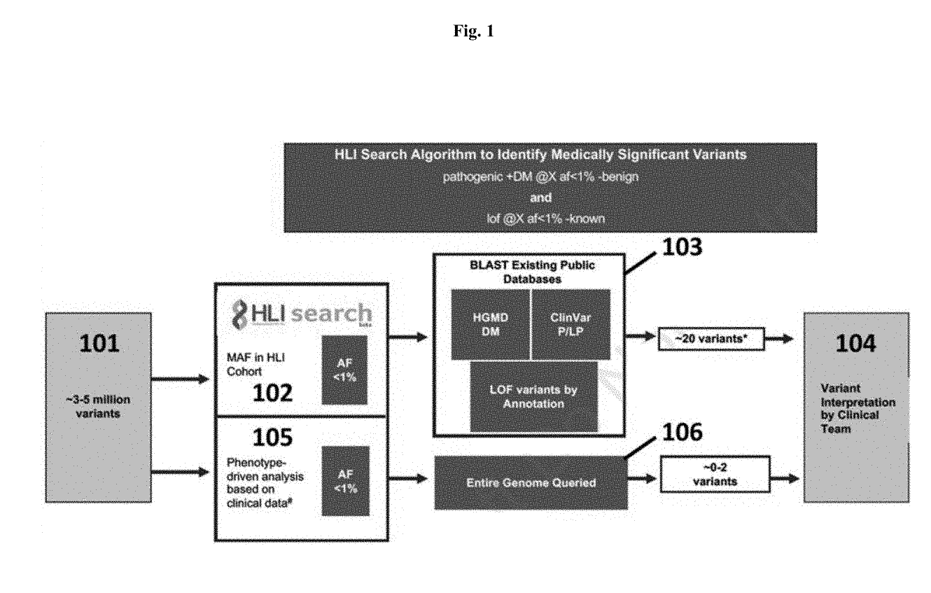

Referring to FIG. 1 an individual's GSVs 101 can be selected for those that have population based cutoff in allele frequency 102 (in this case minor allele frequency less than 1%, but alternatively less than 0.5 or 0.1%). These can then be used to query a known or proprietary database of GSV:disease associations 103 then variants that have known associations can be interpreted by an individual with appropriate technical training 104 and a determination is made as to the genetic risk of the GSV and its overall contribution to the presence of an undiagnosed medical condition or increase in risk of developing the medical condition in the future. This step in 104 can also be automated and combined with any of the independent health metrics to derive a confidence interval or likelihood of the presence of an undiagnosed medical condition or increase in risk of developing the medical condition in the future. Alternatively, all of the GSVs can be queried 106 and compared to phenotypic data from individuals with a known medical condition 105. The same step of determining 104 can be conducted as above.

Health Metrics

The methods, computer media, and systems described herein utilize a plurality of health metrics to diagnose diseases and risk factors. The method can deploy 2, 3, 4, 5, 6, 7, 8, 9, 10, 20, 30, 40, 50, 60, 70, 80, 90, 100, 200, or more independent health metrics, including increments therein. A health metric is a discreet result from a diagnostic test determined by a physician, laboratory, specialist, or questionnaire. The health metrics of this disclosure comprise phenotypic or clinical measurements gathered from an individual. The health measurement can be those normally acquired in a physician's office such as height, weight, blood pressure, reflex measurements, skin fold measurements, temperature, blood oxygen saturation, resting pulse rate, urinalysis and the like. The health measurement can be those normally measured by a laboratory using a patient's blood, plasma, serum, saliva, stool, urine, semen, or pap smear and can include: analysis of blood lipids, such as, fatty acids, non-esterified fatty acids, omega-3 fatty acids, cholesterols, high-density lipoprotein (HDL), low-density lipoprotein (LDL), very low-density lipoprotein (VLDL), chylomicrons, triglycerides, diglycerides, monoglycerides; measures of carbohydrate usage, such as glucose levels (fasting or non-fasting), oral glucose tolerance test, h1Ac; liver enzymes and markers, such as, aspartate aminotransferase, alkaline phosphatase, aspartate aminotransferase, albumin, bilirubin; electrolytes, such as, calcium, sodium, potassium, magnesium, chloride; blood pH, bicarbonate, hemoglobin, red cell count, white blood cell count; specific markers for cancer such as, for example, prostate specific antigen; tests for viral infection such as antibodies against or sequences from, HIV, hepatitis A, B, or C; or bacterial or yeast cultures. The health measurement can be more specialized and comprise histological examination, MM analysis, EKG analysis, EEG analysis, cardiac stress test, psychological evaluation, gait and balance test, ultrasound, CAT scan, X-rays, bone density measurements, body composition measurements, colonoscopy, or PET scans. The health measurement can comprise demographic factors such as age, gender, race, genetic origin or ancestry, health history, or family history. The health measurement can also comprise measurements that are tracked by wearable devices, such as a wearable health monitor, including activity, sleep/wake cycle, sleep analysis, pulse rate and rhythm and the like.

### Non-Genetic Risk Rule

The measured health metrics of an individual are used to set that individual's non-genetic risk. This non-genetic risk can be disease specific, for example, diabetes, heart failure, specific cancers, or other mortalities. This non-genetic risk is defined by a non-genetic risk rule. Physiological measurements are ranked and segmented by known distributions of the physiological measurement. For example, LDL in the 75^{th} percentile or above may be used when determining a risk for a congestive heart failure. The non-genetic risk rule can rank physiological measurements by percentile, quartile, quintile, etc. For example, only the top 99^{th}, 98^{th}, 97^{th}, 96^{th}, 95^{th}, 94^{th}, 93^{rd}, 92^{nd}, 91^{st}, 90^{th}, 80^{th}, 80^{th}, 70^{th}, 60^{th}, or 50^{th} percentile, including increments therein, may be used when determining a non-genetic risk. The non-genetic risk can also be defined in part by a binary response such as a Yes or NO on a questionnaire concerning health history, environmental factors, or family history, or by a plus or minus result from for example an MM or EKG test.

The methods described herein for determining risk for and diagnose of medical conditions comprise combining a plurality of health metrics wherein one of the health metrics comprise nucleotide sequence data and at least one other health metric, but can comprise 2, 3, 4, 5, 6, 7, 8, 9, 10, 20, 30, 40, 50, 60, 70, 80, 90, 100, 200, or more independent health metrics, including increments therein. The genetic and non-genetic risk rules are then combined and appropriately weighted per disease. For example the genetic risk can be assigned a weight that is 10%, 20%, 30%, 40%, 50%, 60%, 70%, 80%, 90%, or 100% more, including increments therein, than the non-genetic risk. For example the non-genetic risk can be assigned a weight that is 10%, 20%, 30%, 40%, 50%, 60%, 70%, 80%, 90%, or 100% more, including increments therein, than the genetic risk. For example, the genetic risk can be assigned a weight that is 2-fold, 3-fold, 4-fold, 5-fold, 6-fold, 7-fold, 8-fold, 9-fold, 10-fold, 20-fold, 50-fold, or 100-fold more, including increments therein, than the non-genetic risk. For example, the non-genetic risk can be assigned a weight that is 2-fold, 3-fold, 4-fold, 5-fold, 6-fold, 7-fold, 8-fold, 9-fold, 10-fold, 20-fold, 50-fold, or 100-fold more, including increments therein, than the genetic risk.

### Digital Processing Device

The platforms, systems, media, and methods described herein may include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, and notebook computers. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

The digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

The digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

The digital processing device, in some cases, includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

The digital processing device, in some cases, includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

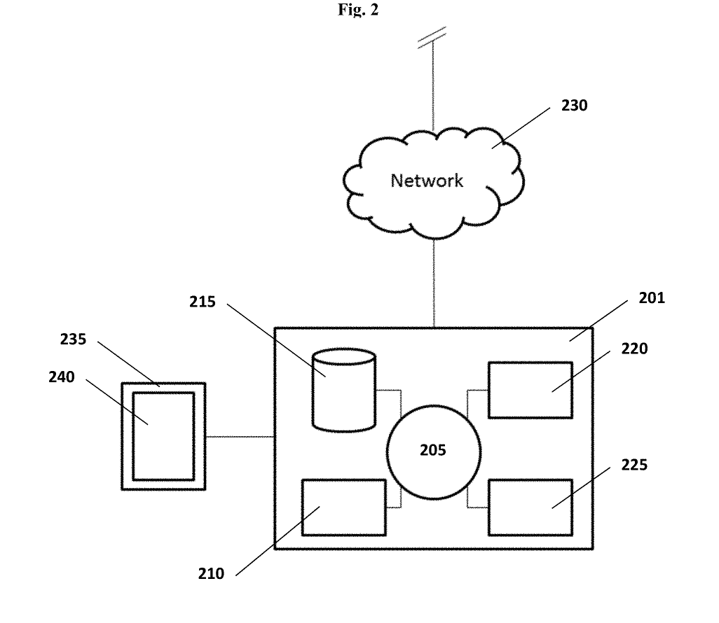

Referring to FIG. 2, in a particular embodiment, an exemplary digital processing device 201 is programmed or otherwise configured to carry out the methods described herein. The device 201 can regulate various aspects of calculating risks for medical conditions, determining treatments, and determining undiagnosed medical conditions of the present disclosure. In this embodiment, the digital processing device 201 includes a central processing unit (CPU, also “processor” and “computer processor” herein) 205, which can be a single core or multi core processor, or a plurality of processors for parallel processing. The digital processing device 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The digital processing device 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the device 201, can implement a peer-to-peer network, which may enable devices coupled to the device 201 to behave as a client or a server.

Continuing to refer to FIG. 2, the CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and write back. The CPU 205 can be part of a circuit, such as an integrated circuit. One or more other components of the device 201 can be included in the circuit. In some cases, the circuit is an application specific integrated circuit (ASIC) or a field programmable gate array (FPGA).

Continuing to refer to FIG. 2, the storage unit 215 can store files, such as drivers, libraries and saved programs. The storage unit 215 can store user data, e.g., user preferences and user programs. The digital processing device 201 in some cases can include one or more additional data storage units that are external, such as located on a remote server that is in communication through an intranet or the Internet.

Continuing to refer to FIG. 2, the digital processing device 201 can communicate with one or more remote computer systems through the network 230. For instance, the device 201 can communicate with a remote computer system of a user. Examples of remote computer systems include personal computers (e.g., portable PC), slate or tablet PCs (e.g., Apple®iPad, Samsung®Galaxy Tab), telephones, Smart phones (e.g., Apple®iPhone, Android-enabled device, Blackberry®), or personal digital assistants.

Methods as described herein can be implemented by way of machine (e.g., computer processor) executable code stored on an electronic storage location of the digital processing device 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

### Non-Transitory Computer Readable Storage Medium

The platforms, systems, media, and methods disclosed herein may include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

The platforms, systems, media, and methods disclosed herein may include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

A computer program described herein may include a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft®SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight. In some embodiments, a web application is written to some extent in a server-side coding language such as Active Server Pages (ASP), ColdFusion, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino. In some embodiments, a web application includes a media player element. In various further embodiments, a media player element utilizes one or more of many suitable multimedia technologies including, by way of non-limiting examples, Adobe®Flash®, HTML 5, Apple® QuickTime®, Microsoft® Silverlight®, Java™, and Unity®.

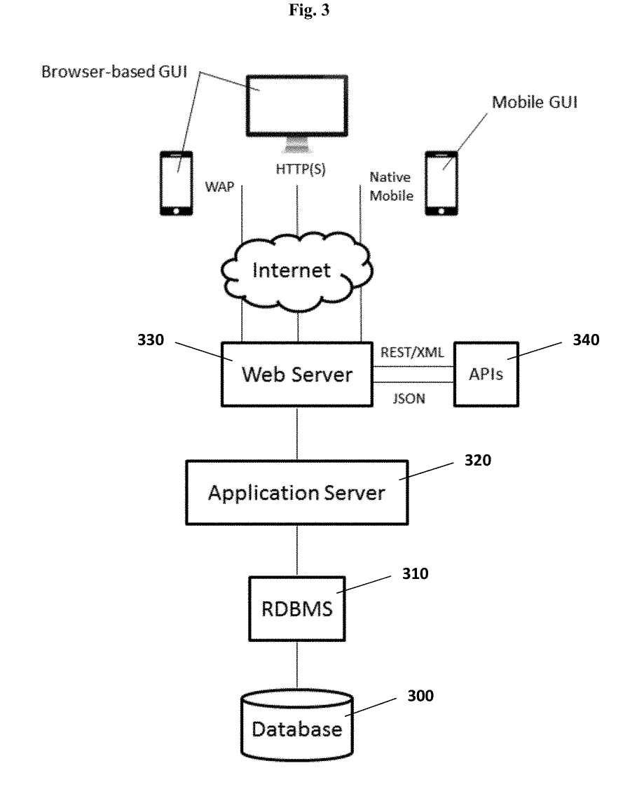

Referring to FIG. 3, in a particular embodiment, an application provision system comprises one or more databases 300 accessed by a relational database management system (RDBMS) 310. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 320 (such as Java servers, .NET servers, PHP servers, and the like) and one or more web servers 330 (such as Apache, IIS, GWS and the like). The web server(s) optionally expose one or more web services via app application programming interfaces (APIs) 340. Via a network, such as the Internet, the system provides browser-based and/or mobile native user interfaces.

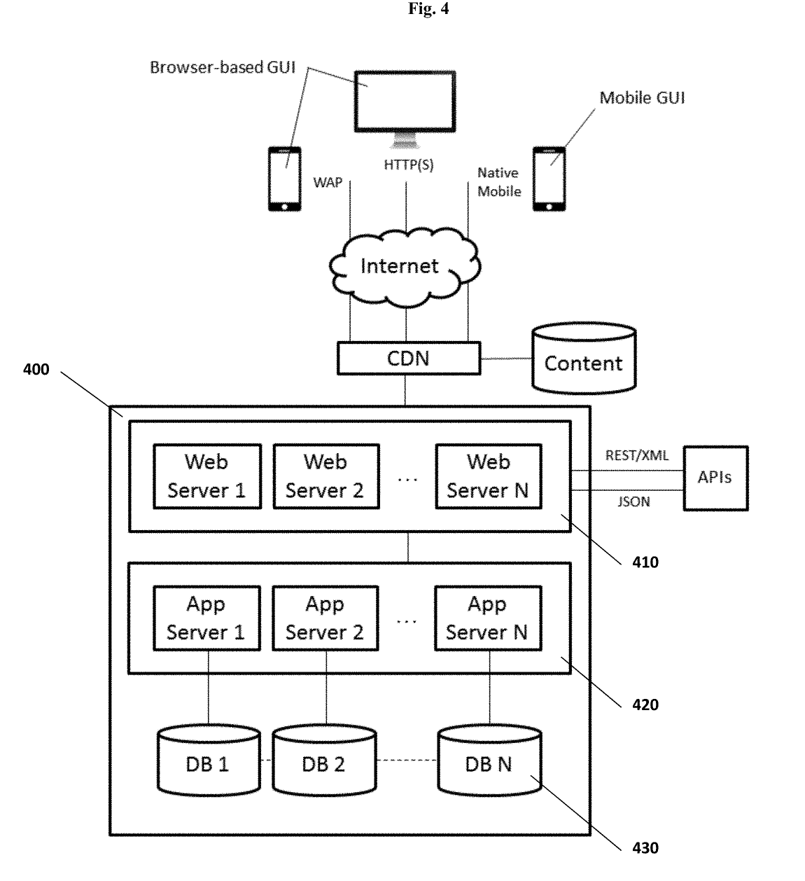

Referring to FIG. 4, in a particular embodiment, an application provision system alternatively has a distributed, cloud-based architecture 400 that comprises elastically load balanced, auto-scaling web server resources 410, application server resources 420 and synchronously replicated databases 430.

### Mobile Application

A computer program described herein may include a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program described herein may include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The platforms, systems, media, and methods disclosed herein may include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. Software modules can be in one computer program or application, or more than one computer program or application. Software modules can be hosted on one machine, or on more than one machine. In further embodiments, software modules are hosted on cloud computing platforms. Software modules can be hosted on one or more machines in one location, one or more machines in more than one location.

### Databases

The platforms, systems, media, and methods disclosed herein may include one or more databases, or use of the same. In view of the disclosure provided herein, those of skill in the art will recognize that many databases are suitable for storage and retrieval of information, such as: genomic data, which may comprise information on genomic sequence variants in a.vcf format or other format; phenotypic data which may comprise physiological measurements, dimensional measurements, health or family histories; and clinical measurements and/or diagnoses which may comprise disease diagnoses or measurements made in a clinical setting. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1

Integrating Genotype and Phenotype Information Uncovers Undiagnosed Conditions and Risk Factors

### Results

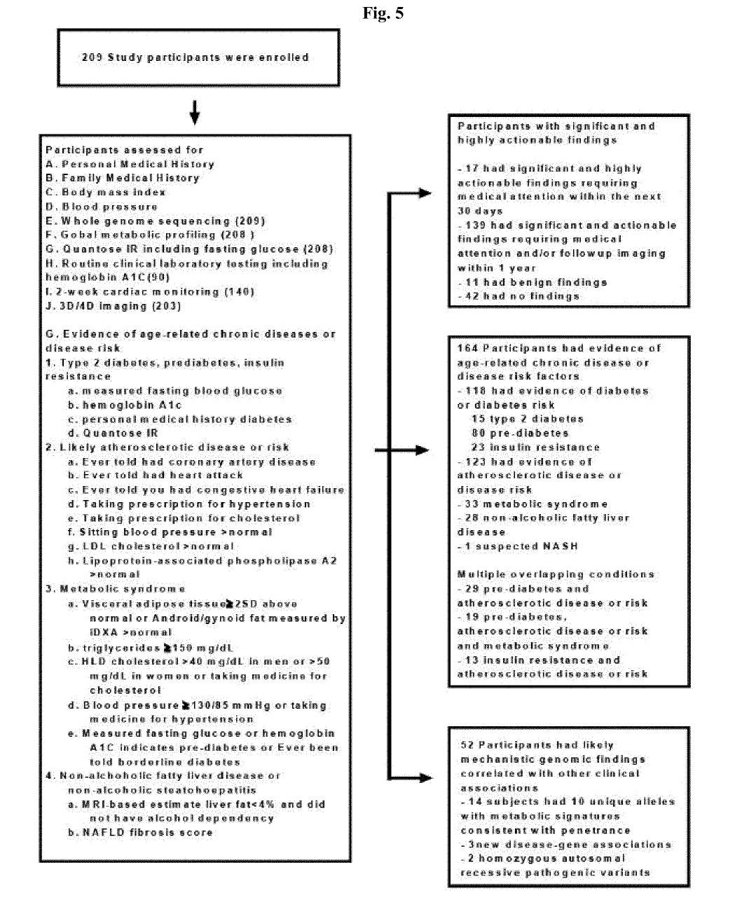

We enrolled 209 study participants, median age 55 yrs., range 20-98 yrs., 34.5% female, between September 10, 2015 and May 16, 2016. Twenty-one (10%) of the 209 participants were from 7 families. Selected characteristics comparing study participants to age and gender-adjusted NHANES cohort, a US population-based sample, is shown in Table 1. Routine clinical laboratory testing was obtained on 90 study participants (43%); Quantose IR (including fasting blood glucose) was obtained on 208 participants and included fasting blood glucose. Magnetic resonance imaging-based quantitative body compartment-specific fat and muscle estimation was conducted on 126 participants (60%). Some portion of the intended 2-week cardiac rhythm monitoring was completed on 140 (67%) participants; the median duration of monitoring was 5.9 days (range 0.8-14 days) (FIG. 5).

TABLE 1
Study Participant Characteristics and Comparison to NHANES

| | | | | | Standardized | 95% | |
|---|---|---|---|---|---|---|---|
| | | | | | Incidence | Confidence | |
| Characteristics | Study Participant | | NHANES Adult | | Ratio 1 | Interval | P-Value |
| Age | | | | | | | 4.43E−40* |
| Median | 55 | | 26 | | | | |
| Range | 20-98 | | 0-80 | | | | |
| Sex | | | | | | | 4.84E−04* |
| Male | 65.6% | | 49.2% | | | | |
| Female | 34.4% | | 50.8% | | | | |
| Measured BMI | | | | | | | |
| Median (25%-75%) | 26 | (23-29) | 24.7 | (20-30) | | | |
| Measured Systolic Blood Pressure | | | | | | | |
| Median (25%-75%) | 123.5 | (115-133) | 116 | (106-128) | | | |
| Measured LDL | | | | | | | |
| Median (25%-75%) | 114.5 | (96-135) | 103 | (81-127) | | | |

| | | | | | |
|---|---|---|---|---|---|
| Diseases | | | | | |
| Neoplasms | | | | | |
| Ever told you had cancer or malignancy | 15.1% | 9.5% | 1.5 | 1.02-2.16 | 3.39E−02* |
| Cardiovascular | | | | | |
| Ever told you had coronary heart disease | 4.1% | 4.0% | 0.9 | 0.38-1.74 | 7.98E−01 |
| Chronic respiratory diseases | | | | | |
| Ever told you had COPD? | 1.0% | 3.3% | 0.2 | 0.02-0.88 | 9.52E−02 |
| Diabetes, urogenital, blood, and | | | | | |
| endocrine diseases | | | | | |
| Doctor told you have diabetes | 4.6% | 7.5% | 0.3 | 0.13-0.54 | 9.63E−04* |
| Cirrhosis and other chronic | | | | | |
| liver diseases | | | | | |
| Ever told you had any liver condition | 6.1% | 4.1% | 1.1 | 0.55-1.89 | 7.75E−01 |
| Neurological disorders | | | | | |
| Blood relatives have Alzheimer's | 13.2% | 13.3% | 1.0 | 0.63-1.44 | 1.00E+00 |
| disease | | | | | |
| Risk Factors | | | | | |
| Alcohol use | | | | | |
| Had at least 12 alcohol drinks/1 yr? | 90.0% | 70.0% | 1.2 | 0.99-1.37 | 2.76E−02* |
| Tobacco smoking | | | | | |
| Smoked at least 100 cigarettes in life | 38.4% | 42.2% | 0.8 | 0.58-0.97 | 8.90E−02 |
| High LDL cholesterol | | | | | |
| Now taking prescribed medicine | 78.9% | 85.4% | 1.1 | 0.74-1.48 | 6.02E−01 |
| High blood pressure | | | | | |
| Ever told you had high blood | 23.0% | 33.7% | 0.5 | 0.38-0.69 | 6.81E−06* |
| pressure | | | | | |
| Taking prescription for hypertension | 73.8% | 83.6% | 0.8 | 0.54-1.14 | 2.44E−01 |

*P ≤ 0.05
1 Giovanni Tripepi, 2010. “Stratification for Confounding-Part 2: Direct and Indirect Standardization”. Nephron Clin Pract
2010; 116: c322-c325
NHANES, National Health and Nutrition Examination Survey, https://www.cdc.gov/nchs/nhanes/

We identified seventeen study participants (8%) with evidence of age-related chronic diseases considered significant and highly actionable requiring prompt medical attention following confirmation of screening findings: four early stage neoplasias (thymoma, renal cell carcinoma, and two high grade prostate neoplasms), one enlarged aortic root, two newly recognized atrial fibrillation cases, two medically significant arrhythmias, one 3^{r}d degree heart block, one primary biliary cholangitis, and one xanthinuria (see Table 2). Some individuals had no detectable genetic risk emphasizing the value of phenotyping technology.

TABLE 2
immediately actionable undiagnosed medical conditions in study participants

| | | | Medical | GBD |
|---|---|---|---|---|
| Sex | Age | Finding | History | Classification |
| M | 67 | Bundle branch block/IVCD; Atrial | no history | Cardiovascular |
| | | fibrillation burden 1% | of CV | diseases |
| | | | disease | |
| M | 57 | Abdomen: 4.8 cm complex left renal cyst | no history | Neoplasm |
| | | with separations. Although this may | cancer | |
| | | represent a complex benign cyst, cystic | | |
| | | renal cell carcinoma is not excluded. | | |
| | | Recommend renal mass protocol CT or | | |
| | | MRI for further evaluation. | | |
| F | 66 | Limited non-contrast neck MRA. String | no history | Cardiovascular |
| | | of beads appearance to the bilateral | CV disease | diseases |
| | | cervical internal carotid arteries may | | |
| | | represent Fibromuscular Dysplasia. | | |
| | | Motion artifact is considered less likely. | | |
| | | Recommend IV contrast enhanced CT or | | |
| | | MR angiogram of the carotid arteries for | | |
| | | further evaluation. | | |
| M | 63 | A 3 cm lobulated contour of the left | History of | Neoplasm |
| | | kidney may represent a benign dromedary | melanoma | |
| | | hump. However there is limited | | |
| | | evaluation. Recommend renal ultrasound | | |
| | | to rule out a mass. | | |
| M | 47 | Right common iliac artery aneurysm | no history | Cardiovascular |
| | | measuring 2.6 cm. No involvement of | CV disease | diseases |
| | | abdominal aorta, internal or external iliac | | |
| | | arteries. Recommend CT angiogram for | | |
| | | further evaluation and confirmation. | | |
| M | 56 | 5 cm anterior mediastinal mass with | no history | Neoplasm |
| | | differential including lymphoma, | cancer; | |
| | | thymoma or germ cell tumor. The mass is | G*P = cardiac | |
| | | most likely a thymoma, likely stage 1 or 2. | hypertrophy | |
| | | There is no obvious vascular invasion and | | |
| | | no lymph nodes. Recommend contrast | | |
| | | enhanced CT and thoracic surgery | | |
| | | consultation. | | |
| F | 47 | A 5 mm aneurysm originating from the | High | Cardiovascular |
| | | left cavernous ICA just proximal to the | cholesterol; | diseases |
| | | ophthalmic artery. Recommend | currently | |
| | | Neurosurgery evaluation. Consider CT or | taking | |
| | | conventional angiogram. | meds | |
| F | 61 | Head: Non-contrast brain MRA. 50% loss | no history | Cardiovascular |
| | | of signal of the left internal carotid artery | CV disease | diseases |
| | | at the junction of the cavernous and | | |
| | | petrous portions may represent artifact | | |
| | | versus partial narrowing. Recommend CT | | |
| | | angiogram of the brain for further | | |
| | | evaluation. | | |
| | | Chest: 4 × 2 cm lesion in the medial right | | |
| | | lower lobe. Suggestion of connection to | | |
| | | the pulmonary vessels. Recommend CT | | |
| | | chest to rule out pulmonary AVM. Other | | |
| | | considerations include mass, sarcoid, | | |
| | | sequestration or atelectasis. | | |
| M | 33 | 1.5 × 1.9 cm cystic lesion in the left | no history | Neoplasm |
| | | parotid gland with differential including | cancer | |
| | | sialocele, pleomorphic adenoma, | | |
| | | lymphatic malformation, or first branchial | | |
| | | cleft cyst. Favor a pleomorphic adenoma. | | |
| | | Recommend contrast enhanced MRI or | | |
| | | CT for further evaluation along with ENT | | |
| | | surgery consultation. | | |
| M | 70 | A 2.5 cm complex lesion in the lower pole | History | Neoplasm |
| | | of the left kidney with differential | skin cancer | |
| | | considerations including hemorrhagic | non-melanoma | |
| | | cyst. A solid mass is not entirely | | |
| | | excluded. Recommend ultrasound or | | |
| | | contrast enhanced CT for further | | |
| | | evaluation. | | |
| F | 69 | Alkaline Phosphatase, S 229 (Abnormal Flag: H) | f/u | Cirrhosis and |
| | | ALT (SGPT) 77 (Abnormal Flag: H) | diagnosis | chronic liver |
| | | AST (SGOT) 73 (Abnormal Flag: H) | of PBC; | disease |
| | | C-Reactive Protein, Quant 7 (Abnormal Flag: H) | G*P = | |
| | | CA 19-9 101 (Abnormal Flag: H) | diabetes | |
| | | Cancer Antigen (CA) 125 58.5 (Abnormal Flag: H) | | |
| | | Cholesterol, Total 253 (Abnormal Flag: H) | | |
| | | Cystatin C 1.05 (Abnormal Flag: H) | | |
| | | Ferritin, Serum 209 (Abnormal Flag: H) | | |
| | | Fibrinogen Antigen 366 (Abnormal Flag: H) | | |
| | | GGT 515 (Abnormal Flag: H) | | |
| | | Iron, Serum 161 (Abnormal Flag: H) | | |
| | | LDL Cholesterol Calc 152 (Abnormal Flag: H) | | |
| | | Lp-PLA2 386 (Abnormal Flag: H) | | |
| | | Platelets 130 (Abnormal Flag: L) | | |
| M | 68 | Paroxysmal atrial fibrillation; Atrial | Report incr | Cardiovascular |
| | | fibrillation burden 3% [f/u patient now on | BP no Rx; | diseases |
| | | anticoagulant] | incr chol + | |
| | | | Rx | |
| M | 73 | First degree AV block; Atrial fibrillation | Report incr | Cardiovascular |
| | | burden 7% [f/u patient now on | BP + Rx | diseases |
| | | anticoagulant] | | |
| F | 64 | Atrial fibrillation burden 2% | Report incr | Cardiovascular |
| | | | Chol + Rx; | diseases |
| | | | incr BP no Rx | |
| M | 65 | Two lesions as noted, the most concerning | No history | Neoplasm |
| | | of which is a PIRADS 4 lesion in the left | cancer | |
| | | posterior peripheral zone. Recommend | | |
| | | urology consultation to consider targeted | | |
| | | biopsy. The anterior transitional zone | | |
| | | lesion is a PIRADS 3 lesion which may | | |
| | | represent a BPH nodule although | | |
| | | neoplasia is within the differential. | | |
| M | 69 | Prostate volume of [52] cc. (normal range | F/u finding | Neoplasm |
| | | is 15-30 cc). | of prostate | |
| | | Right lateral peripheral/transitional zone | cancer | |
| | | lesion as noted above is stable going back | | |
| | | two exams from Jan. 4, 2014 and Jan. 9, 2013. The | | |
| | | lesion is categorized as PIRADS 3. Favor | | |
| | | BPH nodule given stability, but neoplasia | | |
| | | is within the differential. Recommend | | |
| | | close imaging follow up. | | |
| M | 66 | Xanthinuria on Metabolome analysis | History of | |
| | | [xanthine kidney stones] | kidney | |
| | | | stones (6) | |

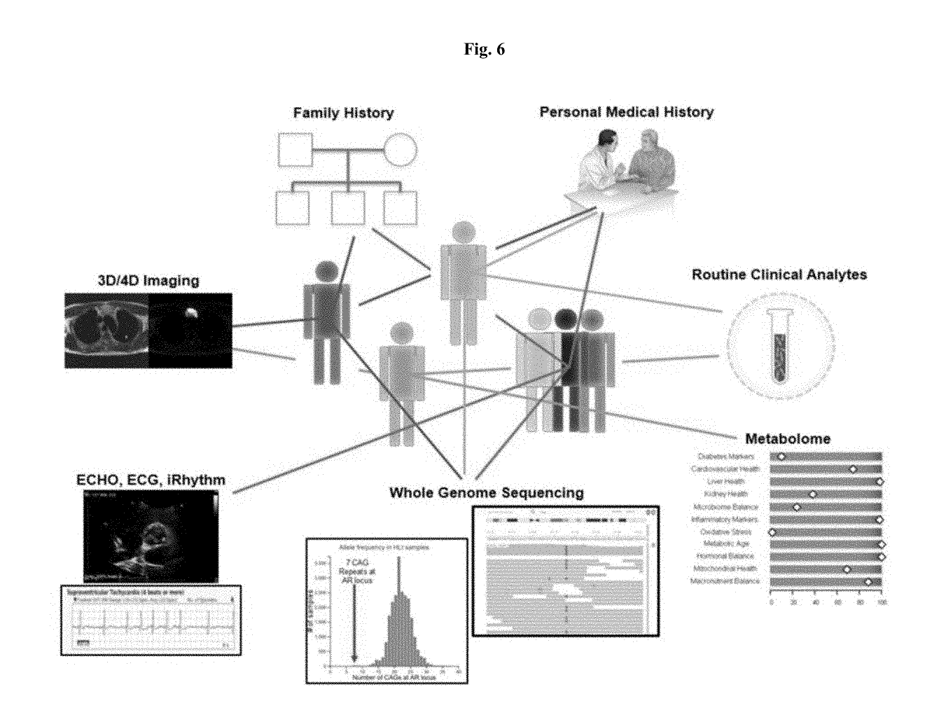

Table 3 lists the pathogenic associations of genomic variants. 52 (25%, 1:4) participants had likely mechanistic genotype-phenotype associations (FIG. 6). Of the 52 variants there were 34 unique genes38 unique variants, zygosity was 50 heterozygous and 2 homozygous, with 3 new variant-disease associations observed in 2 different families.

TABLE 3
Pathogenic associations from this study

| | | | | | | |
|---|---|---|---|---|---|---|
| | | Disease Associated | | | | Personal Medical History/ |
| GBD | Gene | with Gene Variant | MOI | c.HGVS | Zygosity | Family Medical History |

Pathogenic

| | | | | | | |
|---|---|---|---|---|---|---|
| Neoplasm | RAD50 | Hereditary cancer- | AD | c.326_329 | het | FH: maternal family has |
| | | predisposing | | delCAGA | | clustering of cancer, at least |
| | | syndrome | | | | 2 cases of colon cancer |
| Neoplasm | NBN | Hereditary cancer- | AD | c.657_661 | het | FH: father's sibling with |
| | | predisposing | | delACAAA | | brain cancer |
| | | syndrome | | | | |
| Neoplasm | NBN | Hereditary cancer- | AD | c.127C>T | het | FH: brother with carcinoid |
| | | predisposing | | | | tumor, mother with |
| | | syndrome | | | | glioblastoma multiforme, |
| | | | | | | maternal aunt with breast |
| | | | | | | cancer, father with |
| | | | | | | amelanotic melanoma, |
| | | | | | | paternal uncle with an |
| | | | | | | unspecified type of cancer, |
| | | | | | | paternal uncle with |
| | | | | | | nasopharyngeal carcinoma |
| Neoplasm | ALDH2 | Aldehyde | AD | c.1510G>A | het | FH: father with renal cell |
| | | dehydrogenase | | | | cancer, brother died of |
| | | deficiency, | | | | esophageal cancer, Primary |
| | | susceptibility to | | | | bile acids were elevated |
| | | esophageal cancer | | | | indicative of liver |
| | | | | | | dysfunction. |
| | | | | | | Methionine sulfoxide and |
| | | | | | | cysteine-glutathione |
| | | | | | | disulfide were greatly |
| | | | | | | elevated indicative of |
| | | | | | | oxidative stress. Reduced |
| | | | | | | cysteinylglycine and 5- |
| | | | | | | oxoproline were suggestive |
| | | | | | | of limited glutathione |
| | | | | | | synthesis. |
| Neoplasm | ALDH2 | Aldehyde | AD | c.1510G>A | het | FH: paternal grandfather |
| | | dehydrogenase | | | | died of renal cell cancer, |
| | | deficiency, | | | | paternal uncle died of |
| | | susceptibility to | | | | esophageal cancer, paternal |
| | | esophageal cancer | | | | grandfather's siblings died |
| | | | | | | of esophageal cancer, liver |
| | | | | | | fat: 26% ALT is high |
| | | | | | | Extremely reduced 5- |
| | | | | | | oxoproline, and cysteine |
| | | | | | | suggested that glutathione |
| | | | | | | metabolism was impacted. |
| | | | | | | Greatly elevated cysteine- |
| | | | | | | glutathione disulfide was |
| | | | | | | indicative of oxidative |
| | | | | | | stress. |
| Neoplasm | ALDH2 | Aldehyde | AD | c.1510G>A | het | FH: paternal grandfather |
| | | dehydrogenase | | | | died of renal cell cancer, |
| | | deficiency, | | | | paternal uncle died of |
| | | susceptibility to | | | | esophageal cancer, paternal |
| | | esophageal cancer | | | | grandfather's siblings died |
| | | | | | | of esophageal cancer, |
| | | | | | | Greatly reduced cysteine, |
| | | | | | | cysteine sulfinic acid, 5- |
| | | | | | | oxoproline and |
| | | | | | | cysteinylglycine suggested |
| | | | | | | that glutathione metabolism |
| | | | | | | was impacted. Liver fat: |
| | | | | | | 5% ALT: Slightly higher |
| Neoplasm | ATM | Hereditary cancer- | AD, | c.6100C>T | het | PH: Colon cancer |
| | | predisposing | AR | | | FH: Maternal grandmother |
| | | syndrome, Ataxia | | | | with lung cancer, father |
| | | telangiectasia, | | | | with mantle cell lymphoma |
| | | | | | | and renal cell carcinoma, |
| | | | | | | paternal grandmother |
| | | | | | | deceased from leukemia |
| Cardiovascular | PKP2 | Arrhythmogenic | AD | c.314delC | het | PH: history of a mitral |
| | | right ventricular | | | | valve tear; |
| | | dysplasia 9 | | | | ECG: Sinus bradycardia |
| | | | | | | with 1st degree AV block. |
| | | | | | | Rightward axis, incomplete |
| | | | | | | right bundle branch block, |
| | | | | | | borderline ECG; |
| | | | | | | Echo: mild concentric left |
| | | | | | | ventricular hypertrophy, |
| | | | | | | mild enlargement of the left |
| | | | | | | atrium; iRhythm: |
| | | | | | | Arrhythmia: |
| | | | | | | Supraventricular |
| | | | | | | tachycardia, 6 episodes. |
| | | | | | | FH: father and paternal |
| | | | | | | grandfather had myocardial |
| | | | | | | infarction |
| Cardiovascular | APOB | Familial | AD | c.10580G>A | het | LabCorp: high cholesterol, |
| | | hypercholesterolemia | | | | LDL |
| | | | | | | Echo: mild concentric left |
| | | | | | | ventricular hypertrophy FH: |
| | | | | | | first degree relative with |
| | | | | | | atherosclerosis, maternal 1st |
| | | | | | | and 2nd degree relatives |
| | | | | | | have cardiac problems |
| Cirrhosis | HFE | Hemochromatosis; | AR | c.845G>A | homo | FH: sister with hereditary |
| | | susceptibility to | | | | hemochromatosis PH: |
| | | cirrhosis, diabetes | | | | possible hereditary |
| | | and liver cancer | | | | hemochromatosis and |
| | | | | | | diabetes (on metformin), |
| | | | | | | iRhythm: 1 episode of |
| | | | | | | ventricular tachycardia and |
| | | | | | | supraventricular |
| | | | | | | tachycardia, Echo: mild |
| | | | | | | concentric left ventricular |
| | | | | | | hypertrophy, mild |
| | | | | | | enlargement of left ventricle |
| | | | | | | cavity, and a focal high |
| | | | | | | signal echodensity on the |
| | | | | | | aortic valve which does not |
| | | | | | | have independent mobility. |
| | | | | | | ECG: Right bundle branch |
| | | | | | | block, left anterior |
| | | | | | | fascicular block, |
| | | | | | | Metabolon: impaired |
| | | | | | | glucose tolerance, MRI: |
| | | | | | | Liver iron level is normal |
| | | | | | | (47 Hz) |
| Diabetes | SPINK1 | Pancreatitis; | AR, AD | c.101A>G | het | Metabolic markers indicate |
| | | Susceptibility to | | | | impaired insulin sensitivity |
| | | fibrocalculous | | | | |
| | | pancreatic diabetes, | | | | |
| | | Tropical calcific | | | | |
| | | pancreatitis | | | | |
| Diabetes | SPINK1 | Pancreatitis; | AR, AD | c.101A>G | het | Metabolic markers showed |
| | | Susceptibility to | | | | impaired insulin sensitivity |
| | | fibrocalculous | | | | |
| | | pancreatic diabetes, | | | | |
| | | Tropical calcific | | | | |
| | | pancreatitis | | | | |
| Diabetes | SPINK1 | Pancreatitis; | AR, | c.101A>G | het | Metabolic markers showed |
| | | Susceptibility to | AD | | | significant insulin |
| | | fibrocalculous | | | | resistance, MRI: two 6 mm |
| | | pancreatic diabetes, | | | | cystic lesions in pancreas, |
| | | Tropical calcific | | | | LabCorp: CA 19-9 is |
| | | pancreatitis | | | | significantly high, Cancer |
| | | | | | | Antigen 125 is high |
| | | | | | | Metabolic markers involved |
| | | | | | | inflammatory are high FH: |
| | | | | | | brother with diabetes |
| Diabetes | SPINK1 | Pancreatitis; | AR, AD | c.101A>G | het | LabCorp: glucose is high |
| | | Susceptibility to | | | | Metabolic markers |
| | | fibrocalculous | | | | indicated impaired glucose |
| | | pancreatic diabetes, | | | | tolerance and impaired |
| | | Tropical calcific | | | | insulin sensitivity |
| | | pancreatitis | | | | |
| Diabetes | FMO3 | Trimethyla minuria | AR | c.458C>T | homo | PH: fishy odor, increased |
| | | | | | | branch chain amino acid |
| | | | | | | metabolite markers |
| Metabolic | ACAD M | Medium-chain | AR | c.1084A>G | het | Medium chain |
| | | acyl-coenzyme A | | | | acylcarnitines were greatly |
| | | dehydrogenase | | | | elevated and BHBA levels |
| | | deficiency | | | | low. |
| Metabolic | ACADS | Deficiency of | AR | c.319C>T, | het | Butyrylcarnitine and |
| | | butyryl-CoA | | c.511C>T* | | ethylmalonate were both |
| | | dehydrogenase | | | | extremely elevated. |
| Metabolic | ACSF3 | Combined malonic | AR | c.1672C>T | het | Malonylcarnitine and 2- |
| | | and methylmalonic | | | | methylmalonylcarnitine |
| | | aciduria | | | | were greatly elevated |
| Metabolic | ALDH2 | Aldehyde | AD | c.1510G>A | het | Reduced cysteinylglycine |
| | | dehydrogenase | | | | and 5-oxoproline were |
| | | deficiency, | | | | suggestive of impaired |
| | | susceptibility to | | | | glutathione metabolism. |
| | | esophageal cancer | | | | Cysteine-glutathione |
| | | | | | | disulfide was greatly |
| | | | | | | elevated indicative of |
| | | | | | | oxidative stress. |
| Metabolic | ALDH2 | Aldehyde | AD | c.1510G>A | het | Extremely reduced 5- |
| | | dehydrogenase | | | | oxoproline and cysteine but |
| | | deficiency, | | | | greatly elevated cysteine |
| | | susceptibility to | | | | suggested that glutathione |
| | | esophageal cancer | | | | metabolism was impacted. |
| | | | | | | Liver fat: 5% |
| Metabolic | CTH | Cystathioninuria | AR | c.200C>T | het | Cystathionine was greatly |
| | | | | | | elevated. |
| Metabolic | PAH | Phenylketonuria | AR | c.814G>T | het | Phenylalanine was high |
| | | | | | | extreme and tyrosine was |
| | | | | | | low. |

Likely Pathogenic

| | | | | | | |
|---|---|---|---|---|---|---|
| Neoplasm | EPCAM | Lynch syndrome | AD | c.491 + 1G>A | het | FH: Father with leukemia, |
| | | | | | | maternal grandmother with |
| | | | | | | brain tumor, maternal great |
| | | | | | | aunt with liver cancer and |
| | | | | | | maternal great aunt with |
| | | | | | | brain cancer |
| Neoplasm | TP53 | Osteosarcoma, Li | AD | c.844C>T | het | PH: CLL Dx at 2013, |
| | | Fraumeni-like | | | | prostate cancer Dx at 1997, |
| | | syndrome | | | | basal cell carcinoma and |
| | | | | | | squamous cell carcinoma |
| | | | | | | PH: 1st degree relative with |
| | | | | | | 2 breast primaries (early |
| | | | | | | onset in 40s), another first |
| | | | | | | degree relative with |
| | | | | | | Hodgkins lymphoma (client |
| | | | | | | believes this was acquired |
| | | | | | | however). Question of |
| | | | | | | Non-Hodgkins lymphoma |
| | | | | | | in another 1st degree |
| | | | | | | relative |
| Neoplasm | RECQL | Hereditary cancer- | AD | c.643C>T | het | FH: mother with possibly |
| | | predisposing | | | | ovarian cancer, maternal |
| | | syndrome | | | | uncle with lung cancer, |
| | | | | | | maternal aunt with |
| | | | | | | unknown cancer, maternal |
| | | | | | | uncle with possibly |
| | | | | | | colorectal cancer, maternal |
| | | | | | | aunt with breast cancer, |
| | | | | | | maternal grandfather with |
| | | | | | | unknown cancer, father |
| | | | | | | with prostate and bladder |
| | | | | | | cancer |
| Cardiovascular | KCNH2 | Long QT syndrome | AD | c.2785dup G | het | PH: Non-specific T wave |
| | | | | | | abnormality, borderline |
| | | | | | | prolonged QT interval, |
| | | | | | | abnormal ECG (ECG) FH: |
| | | | | | | brother with atrial |
| | | | | | | fibrillation iRhythm: |
| | | | | | | negative |
| Other | TNFRSF13B | Common variable | AR, AD | c.310T>C | het | MRI: mildly enlarged |
| | | immunodeficiency 2 | | | | periportal lymph node, |
| | | | | | | mildly enlarged spleen |
| | | | | | | LabCorp: elevated ALT, |
| | | | | | | AST, Alkaline phosphatase, |
| | | | | | | Fibrinogen antigen, GGT, |
| | | | | | | CA19-9, CA125, Cystaintin |
| | | | | | | C, Metabolitic markers |
| | | | | | | indicate impaired glucose |
| | | | | | | tolerance, insulin |
| | | | | | | sensitivity, kidney function |
| | | | | | | and elevated levels in |
| | | | | | | metabolites of bile acid and |
| | | | | | | inflammation |
| Metabolic | ASS1 | Citrullinemia | AR | c.1030C>T | het | Arginine, citrulline and N- |
| | | | | | | acetylcitrulline were |
| | | | | | | elevated and urea was very |
| | | | | | | low. |
| Metabolic | GCDH | Glutaricaciduria | AR | c.1093G>A | het | Glutarylcarnitine is |
| | | | | | | extremely high |
| Metabolic | PKLR | Pyruvate kinase | AR | c.1456C>T | het | Extremely elevated glucose, |
| | | deficiency | | | | elevated citrate and elevated |
| | | | | | | heme possibly indicating |
| | | | | | | red blood cell breakdown. |
| Metabolic | SLC7A 9 | Cystinuria | AR | c.544G>A | het | Plasma cysteine extremely |
| | | | | | | low. Requires urine for |
| | | | | | | confirmation. |

Risk Factor

| | | | | | | |
|---|---|---|---|---|---|---|
| Neurologic | APOE | Alzheimer's | AD | c.388T>C | homo | PH: Several mental fog |
| | | disease | | | | recently FH: Father with |
| | | | | | | Alzheimer's disease |
| | | | | | | diagnosed age 84, died of |
| | | | | | | stroke age 86. Paternal |
| | | | | | | grandmother and |
| | | | | | | grandfather with late onset |
| | | | | | | Alzheimer's disease, both |
| | | | | | | died in 80's. |

VUS, VUS-suspicious

| | | | | | | |
|---|---|---|---|---|---|---|
| Neoplasm | PALB2 | Familial cancer of | AD | c.508A>G | het | PH: Unilateral BC and |
| | | breast, Hereditary | | | | Unilateral OC (Dx at 32), |
| | | cancer- | | | | two daughters have been |
| | | predisposing | | | | through genetic test on |
| | | syndrome | | | | BRCAs (negative) |
| Neoplasm | PMS1 | Lynch syndrome | AD | c.1888C>T | het | PH: Several colon polyps |
| | | | | | | removed FH: possible |
| | | | | | | cancers, gastric and lung, in |
| | | | | | | paternal grandparent |
| Neoplasm | CHEK2 | Hereditary cancer- | AD | c.190G>A | het | FH: Father with prostate |
| | | predisposing | | | | cancer, half brother with |
| | | syndrome | | | | throat cancer, maternal |
| | | | | | | grandmother had cancer |
| | | | | | | twice (unknown) |
| Neoplasm | RAD50 | Hereditary cancer- | AD | c.2177G>A | het | PH: Breast adenocarcinoma |
| | | predisposing | | | | (left), treated with |
| | | syndrome | | | | lumpectomy and XRT, |
| | | | | | | NED with clear nodes. FH: |
| | | | | | | Sister with leukemia, at 6 y. |
| | | | | | | Father with metastatic lung |
| | | | | | | cancer (tobacco use). |
| | | | | | | Paternal aunt with breast |
| | | | | | | cancer at 35 y had a |
| | | | | | | granddaughter with cancer |
| | | | | | | at 50 y, unknown type. |
| | | | | | | Another paternal aunt with |
| | | | | | | cancer at 63 y, unknown |
| | | | | | | type (tobacco use). |
| | | | | | | Maternal female cousin |
| | | | | | | (once removed) with skin |
| | | | | | | cancer, not otherwise |
| | | | | | | specified. |
| Cardiovascular | DSP | Arrhythmogenic | AD | c.8531G>T | het | PH: dyslipidemia, iRhythm: |
| | | right ventricular | | | | 8 episodes of |
| | | dysplasia | | | | supraventricular |
| | | | | | | tachycardia, Father |
| | | | | | | deceased at age 83 from |
| | | | | | | myocardial infarction and |
| | | | | | | had a history of congestive |
| | | | | | | heart failure and bundle |
| | | | | | | branch block. He had |
| | | | | | | pacemaker. Mother with a |
| | | | | | | history of a transient |
| | | | | | | ischemic attack in her 60 s. |
| | | | | | | Paternal grandfather with |
| | | | | | | likely heart attack. Paternal |
| | | | | | | grandfather with likely |
| | | | | | | heart attack. Maternal |
| | | | | | | grandmother deceased at |
| | | | | | | age 65 from a stroke. |
| | | | | | | Maternal grandfather with a |
| | | | | | | history of peripheral |
| | | | | | | vascular disease. |
| | | | | | | Maternal aunt with stroke in |
| | | | | | | 50's. |
| Cardiovascular | DSP | Arrhythmogenic | AD | c.8531G>T | het | iRhythm: 1 episode of |
| | | right ventricular | | | | supraventricular tachycardia |
| | | dysplasia | | | | Echo: upper limit of left |
| | | | | | | ventricular wall thickness, |
| | | | | | | Father deceased at age 83 |
| | | | | | | from myocardial infarction |
| | | | | | | and had a history of |
| | | | | | | congestive heart failure and |
| | | | | | | bundle branch block. |
| | | | | | | He had pacemaker. Mother |
| | | | | | | with a history of a transient |
| | | | | | | ischemic attack in her 60 s. |
| | | | | | | Paternal grandfather with |
| | | | | | | likely heart attack. Paternal |
| | | | | | | grandfather with likely |
| | | | | | | heart attack. Maternal |
| | | | | | | grandmother deceased at |
| | | | | | | age 65 from a stroke. |
| | | | | | | Maternal grandfather with a |
| | | | | | | history of peripheral |
| | | | | | | vascular disease. |
| | | | | | | Maternal aunt with stroke in |
| | | | | | | 50's. |
| Cardiovascular | DSP | Arrhythmogenic | AD | c.8531G>T | het | PH: dx 59 yr hypertension, |
| | | right ventricular | | | | Periodic heart flutter, Echo: |
| | | dysplasia | | | | Mitral valve mildly |
| | | | | | | thickened. ECG: Left atrial |
| | | | | | | enlargement, borderline |
| | | | | | | ECG, iRhythm: 2 episode |
| | | | | | | of supraventricular |
| | | | | | | tachycardia, 1 episode of |
| | | | | | | ventricular tachycardia, |
| | | | | | | Father deceased at age 83 |
| | | | | | | from myocardial infarction |
| | | | | | | and had a history of |
| | | | | | | congestive heart failure and |
| | | | | | | bundle branch block. He |
| | | | | | | had pacemaker. Mother |
| | | | | | | with a history of a transient |
| | | | | | | ischemic attack in her 60 s. |
| | | | | | | Paternal grandfather with |
| | | | | | | likely heart attack. Paternal |
| | | | | | | grandfather with likely |
| | | | | | | heart attack. Maternal |
| | | | | | | grandmother deceased at |
| | | | | | | age 65 from a stroke. |
| | | | | | | Maternal grandfather with a |
| | | | | | | history of peripheral |
| | | | | | | vascular disease. Maternal |
| | | | | | | aunt with stroke in 50's. |
| Cardiovascular | APOB | Familial | AD | c.9452C>T | het | PH: Elevated cholesterol |
| | | hypercholesterolemia | | | | (on Crestor) and elevated |
| | | | | | | coronary calcium scoring, |
| | | | | | | LabCorp: Cholesterol: |
| | | | | | | 237 mg/dL, LDL cholesterol |
| | | | | | | Calc: 154 mg/dL (range: 0-99 mg/dL) |
| | | | | | | Apolipoprotein |
| | | | | | | (A-1): 186 mg/dL (range: |
| | | | | | | 110-180 mg/dL) |
| | | | | | | Apolipoprotein B: 88 mg/dL |
| | | | | | | (range: 0-79 mg/dL), |
| | | | | | | FH: Mother with atrial |
| | | | | | | fibrillation, hypertension |
| | | | | | | and high cholesterol |
| Cardiovascular | APOB | Familial | AD | c.9452C>T | het | FH: Mother with |
| | | hypercholesterolemia | | | | hypertension and |
| | | | | | | dyslipidemia. Father with |
| | | | | | | cerebralvenous |
| | | | | | | malformation and high |
| | | | | | | cholesterol. Paternal |
| | | | | | | grandfather with cardiac |
| | | | | | | valve replacement. Paternal |
| | | | | | | grandmother with atrial |
| | | | | | | fibrillation and history of |
| | | | | | | stroke, hypertension, and |
| | | | | | | high cholesterol. Maternal |
| | | | | | | grandmother with vascular |
| | | | | | | disease. Maternal |
| | | | | | | grandfather with valvular |
| | | | | | | abnormality. LabCorp: |
| | | | | | | Cholesterol, total: 247 mg/dL |
| | | | | | | (range 100-199) |
| | | | | | | Triglycerides: 229 mg/dL |
| | | | | | | (range 0-149) LDL |
| | | | | | | Cholesterol Calc: 157 mg/dL |
| | | | | | | (range 0-99) VLDL |
| | | | | | | Cholesterol Cal: 46 mg/dL |
| | | | | | | (range 5-40) Lp-PLA2: 237 ng/mL |
| | | | | | | (range 131-199) |
| Cardiovascular | APOB | Familial | AD | c.9452C>T | het | FH: Mother with |
| | | hypercholesterolemia | | | | hypertension and |
| | | | | | | dyslipidemia. Father with |
| | | | | | | cerebralvenous |
| | | | | | | malformation and high |
| | | | | | | cholesterol. Paternal |
| | | | | | | grandfather with cardiac |
| | | | | | | valve replacement. Paternal |
| | | | | | | grandmother with atrial |
| | | | | | | fibrillation and history of |
| | | | | | | stroke, hypertension, and |
| | | | | | | high cholesterol. Maternal |
| | | | | | | grandmother with vascular |
| | | | | | | disease. Maternal |
| | | | | | | grandfather with valvular |
| | | | | | | abnormality. LabCorp: |
| | | | | | | Cholesterol, total: |
| | | | | | | 254 mg/dL (range 100-199) |
| | | | | | | LDL Cholesterol Calc: |
| | | | | | | 155 mg/dL (range 0-99) |
| | | | | | | Triglycerides: 189 mg/dL |
| | | | | | | (range 0-149) |
| | | | | | | Apolipoprotein B: 147 mg/dL |
| | | | | | | (range 52-135) |
| | | | | | | Apolipo. B/A-1 Ratio: 0.9 |
| | | | | | | ratio units (range 0-0.7) |
| Cardiovascular | MYBPC3 | Dilated | AD | c.1468G>A | het | Echo: Mild concentric left |
| | | Cardiomyopathy, | | | | ventricular hypertrophy and |
| | | Hypertrophic | | | | mild enlargement of left |
| | | Cardiomyopathy | | | | atrium. FH: Father with |
| | | | | | | hypertension and heart |
| | | | | | | disease. Two brothers with |
| | | | | | | hypertension; one with |
| | | | | | | heart attack in his 40 s. Two |
| | | | | | | sisters with hypertension. |
| | | | | | | Maternal grandfather with |
| | | | | | | heart attack. |
| Cardiovascular | MYL2 | Hypertrophic | AD | c.401A>C | het | PH: History of aortic valve |
| | | cardiomyopathy | | | | insufficiency and cardiac |
| | | | | | | enlargement; congenital |
| | | | | | | bicuspid aortic valve; |
| | | | | | | arrhythmia. ECG: |
| | | | | | | abnormal ECG Echo: Mild |
| | | | | | | concentric left ventricular |
| | | | | | | hypertrophy. Mild |
| | | | | | | enlargement of left ventricle |
| | | | | | | cavity. Bicuspid aortic |
| | | | | | | valve. Mild to moderate |
| | | | | | | aortic valve regurgitation. |
| | | | | | | Thickened mitral valve with |
| | | | | | | trace regurgitation. Dilated |
| | | | | | | IVC with respiratory |
| | | | | | | collapse greater than 50%, |
| | | | | | | consistent with mildly |
| | | | | | | elevated right atrial pressure |
| | | | | | | (8 mmHg). An atrial septal |
| | | | | | | aneurysm is present. |
| | | | | | | iRhythm: 2 episodes of |
| | | | | | | supraventricular tachycardia |
| Cardiovascular | RYR2 | Ventricular | AD | c.1396C>G | het | PH: Cardiac palpitations |
| | | tachycardia, | | | | history, Echo: borderline |
| | | polymorphic | | | | left ventricular hypertrophy. |
| | | | | | | ECG: Sinus bradycardia |
| | | | | | | iRhythm: negative. FH: |
| | | | | | | Father, brother, multiple |
| | | | | | | paternal uncles and aunts, |
| | | | | | | and paternal grandmother |
| | | | | | | with autosomal dominant |
| | | | | | | hypertrophic |
| | | | | | | cardiomyopathy (idiopathic |
| | | | | | | hypertrophic subaortic |
| | | | | | | stenosis) |
| Cardiovascular | MYH7 | Cardiomyopathy | AD | c.29G>C | het | PH: Right bundle branch |
| | | | | | | block, Echo-left ventricular |
| | | | | | | hypertrophy, enlargement |
| | | | | | | of left ventricle cavity, high |
| | | | | | | signal echodensity on the |
| | | | | | | aortic valve, suggesting |
| | | | | | | focal valvular calcification, |
| | | | | | | ECG: bifascicular block, |
| | | | | | | right bundle branch block, |
| | | | | | | left anterior fascicular |
| | | | | | | block, abnormal ECG, |
| | | | | | | iRhythm: 1 episode of |
| | | | | | | ventricular tachycardia and |
| | | | | | | supraventricular tachycardia |
| | | | | | | FH: two maternal uncles |
| | | | | | | and maternal grandmother |
| | | | | | | with myocardial infarction, |
| | | | | | | maternal grandfather with |
| | | | | | | stroke, father with coronary |
| | | | | | | artery bypass and |
| | | | | | | myocardial infarction, |
| | | | | | | paternal uncle with stroke, |
| | | | | | | paternal grandfather with |
| | | | | | | myocardial infarction |
| Cardiovascular | LPL | Combined | AR, | c.286G>C | het | PH: Slightly elevated |
| | | hyperlipidemia, | AD | | | cholesterol, Echo: left |
| | | familial, | | | | ventricular hypertrophy |
| | | Lipoprotein lipase | | | | ECG: possible left atrial |
| | | deficiency | | | | enlargement, borderline |
| | | | | | | ECG FH: father with |
| | | | | | | hypertension, high |
| | | | | | | cholesterol and heart attack |
| | | | | | | at age 70, maternal cousin |
| | | | | | | with heart attack in 50's and |
| | | | | | | maternal grandfather with |
| | | | | | | cardiovascular disease |
| Cardiovascular | MYBPC3 | Hypertrophic | AD | c.1000G>A | het | PH: Hypertension Echo: |
| | | cardiomyopathy; | | | | borderline left ventricular |
| | | Dilated | | | | hypertrophy, mild |
| | | cardiomyopathy | | | | regurgitation in mitral valve |
| | | | | | | FH: mother with |
| | | | | | | hypertension and |
| | | | | | | arrhythmia and father with |
| | | | | | | valvular heart condition |
| Diabetes | PRSS1 | Hereditary | AD | c.107C>G | het | PH: DM type 2, metabolic |
| | | Pancreatitis | | | | markers indicated impaired |
| | | | | | | insulin sensitivity, |
| | | | | | | LabCorp: Lipase high FH: |
| | | | | | | mother, brother with DM |
| | | | | | | type 2, sister with |
| | | | | | | pancreatic cancer |
| Diabetes | PRSS1 | Hereditary | AD | c.107C>G | het | PH: metabolic markers |
| | | Pancreatitis | | | | indicated impaired glucose |
| | | | | | | tolerance and insulin |
| | | | | | | sensitivity FH: mother with |
| | | | | | | DM type 2, maternal |
| | | | | | | grandmother with a history |
| | | | | | | of diabetes and maternal |
| | | | | | | aunt with pancreatic cancer |

We identified 164 (78%, >3:4) participants with evidence of age-related chronic disease or risk factors. One-hundred-and-eighteen study participants (56%) had evidence of diabetes or risk for diabetes: 15 (7%) had type 2 diabetes; 80 (38%) had pre-diabetes (38%), and 23 (11%) had insulin resistance (based on Quantose IR). Only 19 (16%) reported a history of type 2 diabetes or pre- diabetes (Table 1). One-hundred-and-twenty-four participants (59%) had evidence of atherosclerotic disease or risk. Thirty-three (16%) had evidence of metabolic syndrome. Twenty-eight participants (13%) met a screening definition for non-alcoholic fatty liver disease (NAFLD), and one had suspected non-alcoholic steatohepatitis (NASH). Many participants had multiple over-lapping conditions including: 29 with pre-diabetes and atherosclerotic disease or risk; 19 with pre-diabetes, atherosclerotic disease or risk, and metabolic syndrome and; 13 with insulin resistance and atherosclerotic disease or risk (FIG. 5).

We identified 10 unique alleles in 14 subjects with metabolic signatures consistent with penetrance. Metabolic pathways impacted by the allelic differences included fatty acid beta oxidation, fatty acid synthesis, urea cycle, and signatures associated with oxidative stress. Strong metabolic signatures were observed for two polymorphisms matching the genes' function. Two heterozygous ACADS variants, c.1510G>A and c.1030C>T, coding for the short-chain acyl- Coenzyme A dehydrogenase (SCAD) were detected in one case. In another case, the heterozygous ACADM variant c.1456C>T coding for medium-chain acyl-Coenzyme A dehydrogenase (MCAD) was detected and interestingly both enzymes participate in fatty acid beta-oxidation by reducing different fatty acid chain length. SCAD specifically acts on the short chain fatty acid butyryl-CoA and MCAD reduces acyl-CoA chains containing 6-12 carbons. In the absence of SCAD activity, byproducts of butyryl-CoA including butyrycarnitine and ethylmalonate accumulate. Greatly elevated levels of butyrylcarnitine and ethylmalonate (Z-scores above 97.5^{th} percentile) were observed in the plasma suggestive of combined metabolic penetrance of these variants. Moreover, greatly elevated medium chain acyl-carnitines, hexanoylcarnitine, octanoylcarnitine and decanoylcarnitine (Z-scores above 97.5^{th} percentile) were detected suggestive of reduced MCAD activity. Large genome-wide association studies combined with metabolic profiling have previously identified associations between ACADS and MCAD and their respective metabolic substrates lending support to the metabolic penetrance observed on an individual basis in this study. We previously reported on additional metabolomic/genetic variants which are heterozygotes for known recessively inherited disorders. These studies established that “carrier” disease state does not reflect carrier for individual metabolic variation. The number of adult cases of metabolic penetrance will continue to expand using this approach.

Metabolomics analysis also detected xanthinuria in an individual with early onset (20's) recurrent renal stones (6 episodes) as well as the drug effect of xanthine oxidase inhibitors in 3 other individuals. Although hypoxanthine and especially xanthine levels were elevated in both cases, normal urate and elevated orotate and orotidine levels, due to perturbed pyrimidine synthesis , were only observed in individuals taking xanthine oxidase inhibitors (allopurinols) for their gout conditions.

### Health Metric Collection

We enrolled active adults >18 years old (without acute illness, activity-limiting unexplained illness or symptoms, or known active cancer) able to come for 6-8 hours of on-site data collection, were able to undergo magnetic resonance imaging without sedation, in the case of women were not pregnant or attempting to become pregnant, and were interested in undergoing a novel precision medicine screening approach for disease risk detection including genomics and other testing, as part of an institutional review board-approved clinical research protocol. Study results were returned to study participants who were encouraged to involve their primary care physicians.

Participants underwent a verbal review of the institutional review board-approved consent (Western Institutional Review Board) and were given time to ask and receive answers to questions during a one-half to one-hour sessions conducted by health professionals. Study participants underwent standardized activities related to data collection and return of results in pre-visit, visit, and post-visit phases during a 1-year study period.

Selected data were collected regarding past medical and family history, risk factors, and medical symptoms prior to or during study participant visit. Participants were instructed to stop taking supplements for 72 hours, and to fast after dinner the night before their morning appointment. On the day of visit, blood was obtained for whole genome sequencing (Human Longevity, Inc.), global metabolomics and QUANTOSE™ IR (Metabolon), and routine clinical laboratory tests (LabCorp Inc.™) Two-week cardiac rhythm monitoring (Zio XT Patch™, iRhythm Technologies, Inc.™) kits were provided with instructions for use, or monitoring was initiated during visit. Height, weight, and sitting blood pressure were obtained. Genomic variants were annotated using integrated public and proprietary annotation sources in the HLI Knowledgebase including ClinVar, and HGMD (Qiagen). Monogenic rare variants were classified as pathogenic (P), likely pathogenic (LP), or variant of uncertain significance (VUS). The HLI Knowledgebase integrates allele frequencies for variants derived from HU's database of >12,000 sequences and provides a platform for query of these variants with annotation data.

To identify potentially medically significant rare monogenic variants we used an internal version (release 0.27) of HLI Search™ in a two-step process: the first step focused on allele frequency <1% in the HLI cohort with annotation using ClinVar and HGMD as well as predicted loss of function variants; the second step focused on participant-specific phenotype-driven queries using an allele frequency of <1% based on family and individual medical history as well as abnormal clinical testing results. Global metabolic profiling was performed using ultrahigh performance liquid- phase chromatography separation coupled with tandem mass spectrometry to assess the metabolic penetrance of the variants in these subject. Z-scores were calculated for all metabolites in each subject against a reference cohort consisting of 42 fasted subjects of normal health, and metabolites with Z-scores below the 2.5^{th} or above the 97.5^{th} percentiles of the reference cohort were considered to be potentially indicative of metabolic abnormalities that warranted further investigation. Integration of metabolomic and gene sequence data was achieved by a proprietary pathway analysis program developed by Metabolon and HLI.

Study participants underwent whole body magnetic resonance imaging (GE Discovery MR750w 3.0T) in research mode using protocols and post-processing for volumetric brain imaging (Neuroquant™, CorTechs Laboratories™), cancer detection (using restriction spectrum imaging), neurovascular and cardiovascular visualization, liver-specific fat and iron estimation, and quantitative body compartment-specific fat and muscle estimation (AMRA); other post-processing was done by MMIS. GE Lunar iDXA with Pro Package was used for skeletal and metabolic health assessment. Magnetic resonance imaging and iDXA. GE Vivid E95 was used for echocardiography and a GE Mac 2000 was used to obtain a 12-lead resting electrocardiogram. Two-week cardiac monitoring, electrocardiogram, and echocardiography were interpreted by a physician. Participants with likely mechanistic genomic findings correlating with clinical data were identified by expert review to identify convergent genomic and clinical (or phenotype) data relationships including at least two clinical (or phenotype) data elements supporting a genomic observation, including three generation family history and metabolite level correlation based on pathway mapping. Baseline characteristics including reported past medical history for major categories of age-related chronic diseases by study participants were compared to responses from NHANES, a US population-based cohort (Table 1), adjusted for age and sex distributions. Study participants with evidence of age-related chronic diseases considered significant and highly actionable were defined as new genomic and/or other clinical findings which based on current medical practice indicated the need for medical attention to avoid potentially life-threatening consequences immediately or within 30 days from their visit. Participants with evidence of age-related chronic disease or disease risk factors were identified as including: 1) type 2 diabetes, pre-diabetes and insulin resistance (Quantose IR); 2) likely atherosclerotic disease or risk; 3) metabolic syndrome ; 4) non-alcoholic fatty liver disease and non- alcoholic steatohepatitis, based on clinical guidelines or other recent literature. Measured fasting blood glucose, hemoglobin A1C, personal medical history for diabetes, or Quantose IR was used to identify participants as having diabetes, pre-diabetes or insulin resistance. The presence of any of the following were considered to be evidence of likely atherosclerotic disease or risk: “yes” in response to any of the following questions: 1) Ever told you had coronary artery disease, 2) Ever told you had a heart attack, 3) Ever told you had congestive heart failure, 4) Taking prescription for hypertension, and 5) Taking prescription for cholesterol, or if sitting blood pressure >normal, LDL cholesterol>normal, or Lipoprotein-associated phospholipase A2 (Lp-PLA2)>normal. The presence of any three of the following 5 criteria were considered to be evidence of metabolic syndrome: 1) visceral adipose tissue measured by MM (post-processing by AMRA™)>2SD above normal, or android/gynoid fat measured by iDXA >normal; 2) triglycerides>150 mg/dL; 3) HDL cholesterol<40 mg/dL in men and <50 mg/dL in women or the participant is currently taking prescribed medicine for high cholesterol; 4) blood pressure>130/85 mmHg or the participant is currently taking prescription for hypertension; 5) Measured fasting glucose or hemoglobin A1c indicates pre-diabetes or “borderline” in response to the question - Doctor told you have diabetes. The presence of non-alcoholic fatty liver disease or nonalcoholic steatohepatitis were considered likely if: for non-alcoholic fatty liver disease MM-based estimate liver fat was <4% and did not have alcohol dependence, and for these individuals we used a formula including other demographic and laboratory data to identify likely non-alcoholic steatohepatitis.

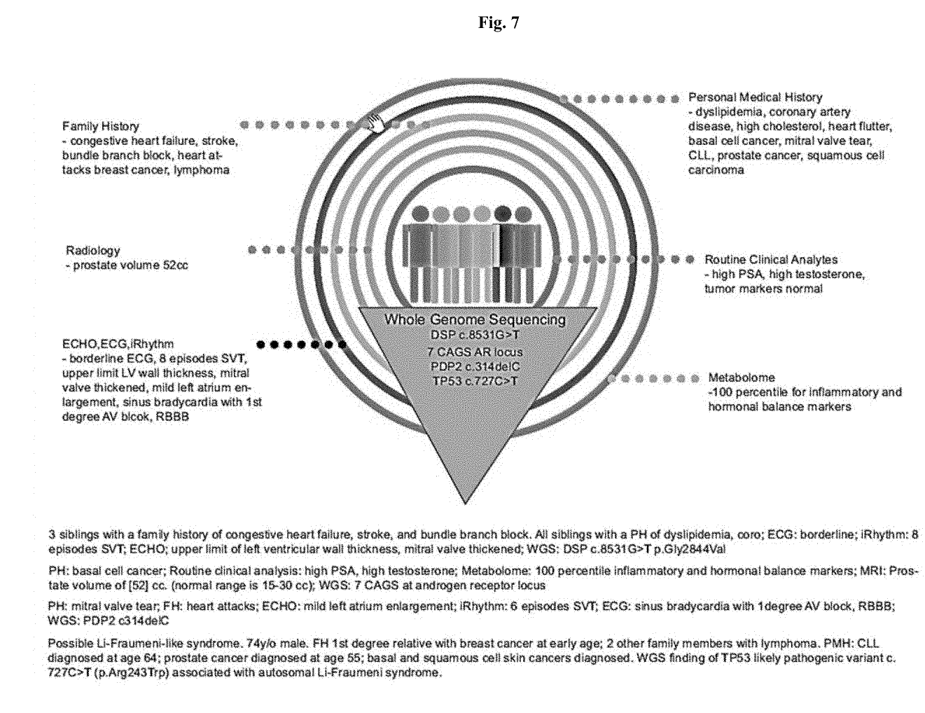

FIG. 6. and FIG. 7 shows phenotype-genotype data integration. Six cases were selected to illustrate the integration of our individual technology data to achieve a precision diagnosis. Case details are found in the legend. This integration requires multiple technology skills and expert medical interpretation. Purple Family History: 1st degree relative with two individuals with breast cancer (early onset in 40s), another first degree relative with Hodgkins lymphoma; Personal Medical History: prostate cancer diagnosed 1997, chronic lymphocytic leukemia diagnosed 2013, basal cell carcinoma and squamous cell carcinoma. Radiology: fMRI revealed focal areas of T1 hypointensity with restricted diffusion in T12, L1, L5 and S2 vertebral bodies likely hemangiomas as findings are stable; Whole Genome Sequencing: TP53 c.844C>T (p.Arg282Trp), a likely pathogenic variant (PMID 19468865, 11370630, 8718514, 21761402, 22672556). Gray Family History: father with elevated cholesterol and elevated coronary calcium scoring, mother with dyslipidemia and hypertension. All grandparents had history of cardiovascular diseases; Routine Clinical Analytes: cholesterol: 247 mg/dL, triglycerides: 229 mg/dL, LDL: 157 mg/dL, VLDL: 46 mg/dL, and Lp- PLA2: 237 ng/mL; Whole Genome Sequencing: APOB c.9452C>T (p.Ser3151Phe), a paternally inherited rare variant. Red Family History: father deceased at age 83 from myocardial infarction and had a history of congestive heart failure and bundle branch block. Mother with a history of a transient ischemic attack in her 60s. Brothers and grandparents had history of high cholesterol, cardiovascular diseases or stroke. Personal Medical History: proband with dyslipidemia and noncritical coronary artery disease from calcium scoring. Cardiovascular: iRhythm showed 8 episodes of supraventricular tachycardia; Whole Genome Sequencing: a rare DSP c.8531G>T (p.Gly2844Val) variant (PMID 20829228) was identified in 3 siblings who also had an abnormal Personal Medical History and abnormal cardiovascular findings. Orange Family History: paternal grandfather with renal cell cancer, paternal grandfather's sibling and paternal uncle with esophageal cancer. Personal Medical History: 31 yrs., BMI 33.2, a bottle of wine per day, Radiology: MRI had shown liver fat at 5%. Routine Clinical Analytes: albumin 5.0 g/dL, AST 481 U/L, GGT 111 IG/L. Metabolome: greatly reduced cysteine, cysteine sulfinic acid, 5-oxoproline and cysteinylglycine suggested that glutathione metabolism was impacted. Whole Genome Sequencing: ALDH2 c.1510G>A (p.Glu504Lys), a pathogenic variant that had been carriers with higher acetaldehyde levels after alcohol consumption and have an increased risk of esophageal cancer (PMID 20010786).

While the preferred embodiments have been shown and described herein, it will be obvious to those skilled in the art that such embodiments are provided by way of example only. Numerous variations, changes, and substitutions will now occur to those skilled in the art without departing from the embodiments disclosed herein. It should be understood that various alternatives to the embodiments described herein may be employed depending on the specific implementations.

What is claimed is:

1. A method of detecting an undiagnosed medical condition:
a) acquiring a plurality of health metrics of an individual, wherein at least one of the plurality of health metrics comprises nucleotide sequence data;
b) implementing a genetic risk rule that defines a genetic risk for the undiagnosed medical condition;
c) implementing a non-genetic risk rule that defines a non-genetic risk for the undiagnosed medical condition; and
d) generating a confidence score for the undiagnosed medical condition that comprises a function of the genetic risk rule and the non-genetic risk rule.

2. The method of claim 1, wherein the undiagnosed medical condition is an increased likelihood of developing a medical condition.

3. The method of claim 1 or 2, wherein the medical condition comprises Parkinson's disease, Alzheimer's disease, ischemic heart disease, hyperlipidemia, high blood pressure, cardiac arrhythmia, long QT syndrome, insulin resistance, Type II diabetes, non-alcoholic fatty liver disease, cirrhosis of the liver, kidney failure, heart failure, depression, bipolar disorder, schizophrenia, or a cancer.

4. The method of claim 3, wherein the cancer comprises breast cancer, prostate cancer, lung cancer, melanoma, pancreatic cancer, kidney cancer, skin cancer, bladder cancer, ovarian cancer, cervical cancer, colon cancer, a leukemia, a lymphoma, head and neck cancer, or brain cancer.

5. The method of any one of claims 1 to 4, wherein the plurality of health metrics further comprises a phenotypic measurement, a family medical history, a personal medical history, or a gut microbiome assessment.

6. The method of claim 5, wherein the phenotypic measurement comprises a clinical measurement or a clinical laboratory test.

7. The method of claim 6, wherein the a clinical measurement or a clinical laboratory test comprises a sleep apnea score, cognitive assessment, neurological test, quantitative Neuro imaging, balance assessment, gait assessment, weight, height, systolic blood pressure, diastolic blood pressure, resting pulse rate, cardiac rhythm monitoring, electrocardiogram, blood lipid levels, blood glucose level, oral glucose tolerance test, blood insulin level, body fat measurement, or whole body MRI.

8. The method of claim 7, wherein the whole body MRI comprises an estimate of total body fat mass or percentage, subcutaneous fat mass or percentage, visceral fat mass or percentage, muscle mass or percentage, liver fat mass or percentage, brain volume, or hippocampal volume.

9. The method of any one of claims 1 to 8, wherein the genetic risk rule comprises ranking a nucleotide sequence variant based upon a score reflecting a pathogenicity of the nucleotide sequence for the undiagnosed medical condition.

10. The method of claim 9, wherein the pathogenicity of the nucleotide sequence for the undiagnosed medical condition is previously determined using a genome wide association study or hazard score associated therewith, presence in ClinVar database, presence in a gene known or suspected to be causative for the undiagnosed medical condition.

11. The method of any one of claims 1 to 10, wherein the second set of rules comprises ranking the non-genetic risk for the undiagnosed medical condition comprises ranking the phenotypic measurement against a plurality of phenotypic measurements derived from a population of individuals.

12. The method of claim 11, wherein ranking the non-genetic risk for the undiagnosed medical condition comprises assigning a quantile score to the non-genetic risk for the undiagnosed medical condition.

13. The method of claim 12, wherein ranking the non-genetic risk for the undiagnosed medical condition comprises assigning a quintile score to the non-genetic risk for the undiagnosed medical condition.

14. The method of any one of claims 1 to 10, wherein the second set of rules comprises determining an amount of standard deviations the phenotypic measurement is away from a mean level for the undiagnosed medical condition derived from a plurality of phenotypic measurements derived from a population of individuals.

15. The method of claim 14, wherein the amount of standard deviations is greater than 2.

16. The method of any one of claims 1 to 15, further comprising delivering a report of the confidence score for the undiagnosed medical condition to a health care provider.

17. The method of any one of claims 1 to 15, further comprising delivering a report of the confidence score for the undiagnosed medical condition to an individual.

18. The method of any one of claims 1 to 16, wherein the undiagnosed medical condition comprises a plurality of undiagnosed medical conditions.

19. A non-transitory computer-readable storage media encoded with a computer program including instructions executable by a processor to create a program to detect an undiagnosed medical condition, comprising instructions for:
a. acquiring a plurality of health metrics of an individual, wherein at least one of the plurality of health metrics comprises nucleotide sequence data;
b. implementing a genetic risk rule that defines a genetic risk for the undiagnosed medical condition;
c. implementing a non-genetic risk rule that defines a non-genetic risk for the undiagnosed medical condition; and
d. generating a confidence score for the undiagnosed medical condition that comprises a function of the genetic risk rule and the non-genetic risk rule.

20. A system for detecting an undiagnosed medical condition, comprising:
one or more processors configured to:
acquire a plurality of health metrics of an individual, wherein at least one of the plurality of health metrics comprises nucleotide sequence data,
implement a genetic risk rule that defines a genetic risk for the undiagnosed medical condition,
implement a non-genetic risk rule that defines a non-genetic risk for the undiagnosed medical condition,
generate a confidence score for the undiagnosed medical condition that comprises a function of the genetic risk rule and the non-genetic risk rule; and
a memory coupled to at least some of the one or more processors, configured to provide the processors with instructions.
